SEQUENCE LISTING (64 sequences)

SEQ ID NO: 1 - protein, 9 aa, artificial sequence
  antibody or linker sequence
FNISVYMMH

SEQ ID NO: 2 - protein, 14 aa, artificial sequence
  antibody or linker sequence
WVASIYPYSGYTYY

SEQ ID NO: 3 - protein, 9 aa, artificial sequence
  antibody or linker sequence
RYVYHALDY

SEQ ID NO: 4 - protein, 10 aa, artificial sequence
  antibody or linker sequence
QRGLRNVAVA

SEQ ID NO: 5 - protein, 11 aa, artificial sequence
  antibody or linker sequence
LLIYSASSLYS

SEQ ID NO: 6 - protein, 9 aa, artificial sequence
  antibody or linker sequence
QQWAVHSLI

SEQ ID NO: 7 - protein, 9 aa, artificial sequence
  antibody or linker sequence
YTFTRYTMH

SEQ ID NO: 8 - protein, 14 aa, artificial sequence
  antibody or linker sequence
WIGYINPSRGYTNY

SEQ ID NO: 9 - protein, 11 aa, artificial sequence
  antibody or linker sequence
RYYDDHYCLDY

SEQ ID NO: 10 - protein, 7 aa, artificial sequence
  antibody or linker sequence
SSVSYMN

SEQ ID NO: 11 - protein, 11 aa, artificial sequence
  antibody or linker sequence
RWIYDTSKVAS

SEQ ID NO: 12 - protein, 8 aa, artificial sequence
  antibody or linker sequence
QQWSSNPL

SEQ ID NO: 13 - protein, 9 aa, artificial sequence
  antibody or linker sequence
YTFTRYTMH

SEQ ID NO: 14 - protein, 14 aa, artificial sequence
  antibody or linker sequence
WIGYINPSRGYTNY

SEQ ID NO: 15 - protein, 11 aa, artificial sequence
  antibody or linker sequence
RYYDDHYCLDY

SEQ ID NO: 16 - protein, 7 aa, artificial sequence
  antibody or linker sequence
SSVSYMN

SEQ ID NO: 17 - protein, 11 aa, artificial sequence
  antibody or linker sequence
RWIYDTSKLAS

SEQ ID NO: 18 - protein, 8 aa, artificial sequence
  antibody or linker sequence
QQWSSNPF

SEQ ID NO: 19 - protein, 118 aa, artificial sequence
  antibody or linker sequence
SEVQLVESGGGLVQPGGSLRLSCAASGFNISVYMMHWVRQAPGKGLEWVASIYPYSGYTYYADSVKGRFTISADTSKNTAYLQMNSLRAEDTAVYYCARYVYHALDYWGQGTLVTVSS

SEQ ID NO: 20 - protein, 111 aa, artificial sequence
  antibody or linker sequence
DIQMTQSPSSLSASVGDRVTITCRASQRGLRNVAVAWYQQKPGKAPKLLIYSASSLYSGVPSRFSGSRSGTDFTLTISSLQPEDFATYYCQQWAVHSLITFGQGTKVEIKR

SEQ ID NO: 21 - protein, 119 aa, artificial sequence
  antibody or linker sequence
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 22 - protein, 106 aa, artificial sequence
  antibody or linker sequence
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 23 - protein, 119 aa, artificial sequence [sequence omitted]

SEQ ID NO: 24 - protein, 107 aa, artificial sequence
  antibody or linker sequence
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 25 - protein, 491 aa, artificial sequence
  antibody or linker sequence
DIQMTQSPSSLSASVGDRVTITCRASQRGLRNVAVAWYQQKPGKAPKLLIYSASSLYSGVPSRFSGSRSGTDFTLTISSLQPEDFATYYCQQWAVHSLITFGQGTKVEIKRGGGGSGGGGSGGGGSEVQLVESGGGLVQPGGSLRLSCAASGFNISVYMMHWVRQAPGKGLEWVASIYPYSGYTYYADSVKGRFTISADTSKNTAYLQMNSLRAEDTAVYYCARYVYHALDYWGQGTLVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 26 - protein, 492 aa, artificial sequence
  antibody or linker sequence
DIQMTQSPSSLSASVGDRVTITCRASQRGLRNVAVAWYQQKPGKAPKLLIYSASSLYSGVPSRFSGSRSGTDFTLTISSLQPEDFATYYCQQWAVHSLITFGQGTKVEIKRGGGGSGGGGSGGGGSEVQLVESGGGLVQPGGSLRLSCAASGFNISVYMMHWVRQAPGKGLEWVASIYPYSGYTYYADSVKGRFTISADTSKNTAYLQMNSLRAEDTAVYYCARYVYHALDYWGQGTLVTVSSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 27 - protein, 244 aa, artificial sequence
  antibody or linker sequence
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 28 - protein, 243 aa, artificial sequence
  antibody or linker sequence
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 29 - protein, 243 aa, artificial sequence
  antibody or linker sequence
DIQMTQSPSSLSASVGDRVTITCRASQRGLRNVAVAWYQQKPGKAPKLLIYSASSLYSGVPSRFSGSRSGTDFTLTISSLQPEDFATYYCQQWAVHSLITFGQGTKVEIKRGGGGSGGGGSGGGGSEVQLVESGGGLVQPGGSLRLSCAASGFNISVYMMHWVRQAPGKGLEWVASIYPYSGYTYYADSVKGRFTISADTSKNTAYLQMNSLRAEDTAVYYCARYVYHALDYWGQGTLVTVSS

SEQ ID NO: 30 - protein, 19 aa, artificial sequence
  antibody or linker sequence
MGWSLILLFLVAVATGVHS

SEQ ID NO: 31 - protein, 14 aa, artificial sequence
  antibody or linker sequence
GGGGSGGGGSGGGG

SEQ ID NO: 32 - protein, 5 aa, artificial sequence
  antibody or linker sequence
GGGGS

SEQ ID NO: 33 - protein, 18 aa, artificial sequence
  antibody or linker sequence
VEGGSGGSGGSGGSGGVD

SEQ ID NO: 34 - protein, 516 aa, artificial sequence
  antibody or linker sequence
MGWSLILLFLVAVATGVHSDIQMTQSPSSLSASVGDRVTITCRASQRGLRNVAVAWYQQKPGKAPKLLIYSASSLYSGVPSRFSGSRSGTDFTLTISSLQPEDFATYYCQQWAVHSLITFGQGTKVEIKRGGGGSGGGGSGGGGSEVQLVESGGGLVQPGGSLRLSCAASGFNISVYMMHWVRQAPGKGLEWVASIYPYSGYTYYADSVKGRFTISADTSKNTAYLQMNSLRAEDTAVYYCARYVYHALDYWGQGTLVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 35 - protein, 7 aa, artificial sequence
  antibody or linker sequence
GFNISVY

SEQ ID NO: 36 - protein, 6 aa, artificial sequence
  antibody or linker sequence
YPYSGY

SEQ ID NO: 37 - protein, 8 aa, artificial sequence
  antibody or linker sequence
YVYHALDY

SEQ ID NO: 38 - protein, 13 aa, artificial sequence
  antibody or linker sequence
RASQRGLRNVAVA

SEQ ID NO: 39 - protein, 7 aa, artificial sequence
  antibody or linker sequence
SASSLYS

SEQ ID NO: 40 - protein, 10 aa, artificial sequence
  antibody or linker sequence
QQWAVHSLIT

SEQ ID NO: 41 - protein, 7 aa, artificial sequence
  antibody or linker sequence
GYTFTRY

SEQ ID NO: 42 - protein, 6 aa, artificial sequence
  antibody or linker sequence
NPSRGY

SEQ ID NO: 43 - protein, 10 aa, artificial sequence
  antibody or linker sequence
YYDDHYCLDY

SEQ ID NO: 44 - protein, 10 aa, artificial sequence
  antibody or linker sequence
RASSSVSYMN

SEQ ID NO: 45 - protein, 7 aa, artificial sequence
  antibody or linker sequence
DTSKVAS

SEQ ID NO: 46 - protein, 9 aa, artificial sequence
  antibody or linker sequence
QQWSSNPLT

SEQ ID NO: 47 - protein, 7 aa, artificial sequence
  antibody or linker sequence
GFNISVY

SEQ ID NO: 48 - protein, 6 aa, artificial sequence
  antibody or linker sequence
YPYSGY

SEQ ID NO: 49 - protein, 8 aa, artificial sequence
  antibody or linker sequence
YVYHALDY

SEQ ID NO: 50 - protein, 13 aa, artificial sequence
  antibody or linker sequence
RASQRGLRNVAVA

SEQ ID NO: 51 - protein, 7 aa, artificial sequence
  antibody or linker sequence
SASSLYS

SEQ ID NO: 52 - protein, 10 aa, artificial sequence
  antibody or linker sequence
QQWAVHSLIT

SEQ ID NO: 53 - protein, 7 aa, artificial sequence
  antibody or linker sequence
GYTFTRY

SEQ ID NO: 54 - protein, 6 aa, artificial sequence
  antibody or linker sequence
NPSRGY

SEQ ID NO: 55 - protein, 10 aa, artificial sequence
  antibody or linker sequence
YYDDHYCLDY

SEQ ID NO: 56 - protein, 10 aa, artificial sequence
  antibody or linker sequence
RASSSVSYMN

SEQ ID NO: 57 - protein, 7 aa, artificial sequence
  antibody or linker sequence
DTSKVAS

SEQ ID NO: 58 - protein, 9 aa, artificial sequence
  antibody or linker sequence
QQWSSNPLT

SEQ ID NO: 59 - protein, 497 aa, artificial sequence
  antibody or linker sequence
DIQMTQSPSSLSASVGDRVTITCRASQRGLRNVAVAWYQQKPGKAPKLLIYSASSLYSGVPSRFSGSRSGTDFTLTISSLQPEDFATYYCQQWAVHSLITFGQGTKVEIKRGGGGSGGGGSGGGGSEVQLVESGGGLVQPGGSLRLSCAASGFNISVYMMHWVRQAPGKGLEWVASIYPYSGYTYYADSVKGRFTISADTSKNTAYLQMNSLRAEDTAVYYCARYVYHALDYWGQGTLVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 60 - protein, 498 aa, artificial sequence
  antibody or linker sequence
DIQMTQSPSSLSASVGDRVTITCRASQRGLRNVAVAWYQQKPGKAPKLLIYSASSLYSGVPSRFSGSRSGTDFTLTISSLQPEDFATYYCQQWAVHSLITFGQGTKVEIKRGGGGSGGGGSGGGGSEVQLVESGGGLVQPGGSLRLSCAASGFNISVYMMHWVRQAPGKGLEWVASIYPYSGYTYYADSVKGRFTISADTSKNTAYLQMNSLRAEDTAVYYCARYVYHALDYWGQGTLVTVSSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRHHHHHH

SEQ ID NO: 61 - protein, 730 aa, artificial sequence
  antibody or linker sequence
SEVQLVESGGGLVQPGGSLRLSCAASGFNISVYMMHWVRQAPGKGLEWVASIYPYSGYTYYADSVKGRFTISADTSKNTAYLQMNSLRAEDTAVYYCARYVYHALDYWGQGTLVTVSSGSSGGDIQMTQSPSSLSASVGDRVTITCRASQRGLRNVAVAWYQQKPGKAPKLLIYSASSLYSGVPSRFSGSRSGTDFTLTISSLQPEDFATYYCQQWAVHSLITFGQGTKVEIKRGGGGSGGGGSGGGGSEVQLVESGGGLVQPGGSLRLSCAASGFNISVYMMHWVRQAPGKGLEWVASIYPYSGYTYYADSVKGRFTISADTSKNTAYLQMNSLRAEDTAVYYCARYVYHALDYWGQGTLVTVSSGGSGGDIQMTQSPSSLSASVGDRVTITCRASQRGLRNVAVAWYQQKPGKAPKLLIYSASSLYSGVPSRFSGSRSGTDFTLTISSLQPEDFATYYCQQWAVHSLITFGQGTKVEIKRGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 62 - protein, 472 aa, artificial sequence
  antibody or linker sequence
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGSGGSSEVQLVESGGGLVQPGGSLRLSCAASGFNISVYMMHWVRQAPGKGLEWVASIYPYSGYTYYADSVKGRFTISADTSKNTAYLQMNSLRAEDTAVYYCARYVYHALDYWGQGTLVTVSSGGSGGSDIQMTQSPSSLSASVGDRVTITCRASQRGLRNVAVAWYQQKPGKAPKLLIYSASSLYSGVPSRFSGSRSGTDFTLTISSLQPEDFATYYCQQWAVHSLITFGQGTKVEIKRGGSGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 63 - protein, 231 aa, artificial sequence
  antibody or linker sequence
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGSGGSSEVQLVESGGGLVQPGGSLRLSCAASGFNISVYMMHWVRQAPGKGLEWVASIYPYSGYTYYADSVKGRFTISADTSKNTAYLQMNSLRAEDTAVYYCARYVYHALDYWGQGTLVTVSSC

SEQ ID NO: 64 - protein, 237 aa, artificial sequence
  antibody or linker sequence
DIQMTQSPSSLSASVGDRVTITCRASQRGLRNVAVAWYQQKPGKAPKLLIYSASSLYSGVPSRFSGSRSGTDFTLTISSLQPEDFATYYCQQWAVHSLITFGQGTKVEIKRGGSGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSC

(19) United States
(12) Patent Application Publication
(10) Pub. No.: US 2022/0315653 A1
(43) Pub. Date: Oct. 6, 2022

(54) BISPECIFIC BINDING AGENT THAT BINDS TO CD117/c-KIT AND CD3

(71) Applicants: UNIVERSITAET ZUERICH, Zurich (CH); ETH ZUERICH, Zurich (CH)

(72) Inventors: Jonathan KIEFER, Zurich (CH); Renier MYBURGH, Aarau (CH); Markus MANZ, Zurich (CH); Dario NERI, Buchs (CH)

(21) Appl. No.: 17/640,412

(22) PCT Filed: Sep. 4, 2020
(86) PCT No.: PCT/EP2020/074807

§ 371 Date: Mar. 4, 2022

(30) Foreign Application Priority Data
Sep. 4, 2019 (GB) GB1912681.2

(51) Int. Cl. C07K 16/28; A61P 35/02

(52) U.S. Cl. CPC C07K 16/2803; C07K 16/2809; A61P 35/02; A61K 2039/505

(57) ABSTRACT

The present invention relates to a bispecific binding agent comprising at least a first binding domain and a second binding domain, wherein said first binding domain binds to CD117/c-KIT and wherein said second binding domain binds to CD3.

### FIELD OF THE INVENTION

The present application relates to a bispecific binding agent that binds to CD117/c-KIT and CD3.

### BACKGROUND

Bispecific antibodies, capable of redirecting T cell activity against target cells, represent a potent class of antibody products in order to achieve a selective biocidal action in vivo. Bispecific antibodies can kill target cells at subnanomolar concentrations in the presence of T cells [15]. One bispecific antibody product (blinatumomab), capable of simultaneous recognition of human CD19 and CD3 antigens, has received marketing authorization for the treatment of Philadelphia chromosome-negative relapsed or refractory B-cell precursor acute lymphoblastic leukemia (ALL) [16]. Blinatumomab is a Bispecific T cell Engager (BiTE™), a recombinant antibody format produced by the sequential fusion of two single-chain Fv antibody fragments [17, 18].

Self-renewing and immature hematopoietic stem and progenitor cells (HSCP) give rise to the myeloid and lymphoid lineage in hematopoiesis [1]. One single hematopoietic stem cell can also be at the origin of hematological malignancies, such as Acute Myeloid Leukemia (AML) and Myelodysplastic Syndrome (MDS) [2, 3]. These conditions are often difficult to treat, because of the difficulty to discriminate between AML and progenitor cells with therapeutic intervention or the risk of myeloablation for particularly aggressive regimens.

The treatment approach featuring preconditioning followed by HSCT may be a life-saving option for some AML patients. High-dose chemotherapy-based conditioning followed by hematopoietic stem cell transplant may lead to cancer cures but is associated with substantial toxicity and a non-negligible mortality rate [4]. This therapeutic procedure often excludes patients who are unfit for aggressive chemotherapeutic regimens (e.g., elderly patient), who may rather receive hypomethylating agents in a palliative setting [5]. Aggressive conditioning regimens may also give rise to a prolonged myelosuppression of AML patients, with potentially negative consequences in terms of their susceptibility to infections [6]. For these reasons, considerable research efforts are being devoted to the discovery of more selective preconditioning strategies and of pharmaceutical agents with potent biocidal activity against AML cells [7, 8].

The selective targeting of CD117 (c-Kit) with monoclonal antibodies has been proposed as a strategy to remove endogenous HSPC (Hematopoietic stem and progenitor cells), enabling an effective but mild pre-conditioning. Irving Weissmann and colleagues have demonstrated that an efficient elimination of HSPCs can be achieved using monoclonal antibodies in IgG format directed against the c-Kit antigen, both in immunocompromised [9] and in immunocompetent [7] mice. This interventional procedure, which can be potentiated by the combination with anti-CD47 antibodies, has recently been translated to the clinical setting using an Anti-Kit Therapeutic Antibody (AMG 191) (TAB-470CL) as a potential therapy for inflammatory diseases, and also potentially enabling a milder conditioning approach before hematopoietic stem cell transplantation in patients with severe combined immunodeficiency (SCID) [10]. The preclinical agent, KTN0182A, is an anti-KIT, pyrrolobenzodiazepine (PBD)-containing antibody-drug conjugate which shows anti-tumor activity in vitro and in vivo against a range of tumor types. However, no such product has so far made it into the marketplace.

It is hence one object of the present invention to improve treatment options for patients suffering from neoplastic diseases.

It is one further object of the present invention to provide further treatment options for patients suffering from a CD117 positive disease.

It is one further object of the present invention to provide further treatment options for patients suffering Acute Myeloid Leukemia (AML) or Myelodysplastic Syndrome (MDS).

These and further objects are met with methods and means according to the independent claims of the present invention. The dependent claims are related to specific embodiments.

### SUMMARY OF THE INVENTION

The present invention provides a bispecific binding agent comprising at least a first binding domain and a second binding domain, wherein said first binding domain binds to CD117/c-KIT and wherein said second binding domain binds to CD3. The invention and general advantages of its features and embodiments will be discussed in detail below.

### BRIEF DESCRIPTION OF THE FIGURES

FIG. 1: Cloning, expression and characterization of CD117×CD3

(A) Chain arrangement of CD117×CD3. The c-Kit antibody 79D [19] was cloned in the chain order VL_{79D}-Linker-VH_{79D} and genetically fused to the humanized version of the anti-CD3 antibody OKT3, called BlinCD3 herein, in the order VH_{BlinCD3}-VL_{BlinCD3}. One exemplary sequence of such bispecific binding agent is shown as SEQ ID NO 25 herein (SEQ ID NO 34 comprises also the signal peptide and the His Tag). The resulting construct was cloned into the mammalian expression vector pcDNA3.1(+). (B) Schematic representation of the CD117×CD3 protein. The N-terminus of the fusion protein is indicated (N′), while a His₆ tag is found at the C-terminus. (C) SDS-PAGE of the purified bispecific antibody showing migration at the expected (monomeric) size of ˜55 kDa. M=Marker, NR=Non-reducing conditions. (D) Mass spectrometric analysis of the purified product showed a single N-glycosylation of 2′053 Dalton, confirming the theoretical mass of 53′475 Da (see also FIG. 7). (E) Binding of CD117×CD3 to recombinant target antigen human c-Kit in a Biacore experiment at the indicated concentrations.

FIG. 2: CD117 expression of target cells and biological in vitro activity of CD117×CD3 against cell lines

(A) FACS profiles of HL60 target cell lines, showing different levels of CD117 expression, which were generated by c-Kit transduction procedures. (B) Quantitative analysis of antigen density on target cell lines. HL60^{low}, HL60^{med}, and HL60^{high} cells were shown to express 7909±960, 25146±2964 and 114597±11450 copies of the target antigen CD117, respectively. (C) Specific lysis of target cells lines is dependent on concentration of CD117×CD3, as well as on target antigen density. Effector (human T cells) to Target (HL60 cell lines with different c-Kit expression level) ratio 10:1. (D) At a fixed CD117×CD3 concentration of 1 μg/ml, effector to target ratio was varied from 1:20 to 20:1.

FIG. 3: Biological in vitro activity of CD117×CD3 in the presence of IL-2

Specific lysis of target cell lines HL60^{low}, HL60^{med}, and HL60^{high} was dependent on concentration of bispecific antibody and antigen density. Addition of IL-2 to boost baseline T Cell activation did not substantially alter lytic activity.

FIG. 4: Biological in vitro activity of CD117×CD3 against primary bone marrow samples and patient AML samples

(A) CD117 expression in three different bone marrow samples from healthy donors (BM #249, BM #333, #BM 351) revealed variable antigen density among cells. (B) Specific lysis of bone marrow samples from (A) in the presence of human T cells and a concentration range of bispecific antibody (long/ml, 100 ng/ml and 1000 ng/ml). (C) Analysis of three different AML patient samples showed CD117 expression variability both within the samples and in an inter-patient comparison (CD45^{dim} blast cells are shown). (D) Specific lysis of blast cell samples from (C) in the presence of autologous T cells in a concentration dependent manner. Effector to Target ratio 10:1 for all 6 samples (B+D). All experiments were performed in triplicates. (E) Example FACS plots of in vitro killing assay for patient sample #878. CD45^{dim} blast cell populations were specifically lysed in a BiTE™-concentration dependent manner.

FIG. 5: Biological in vivo activity of CD117×CD3 against AML cell line in a preventive setting

(A) Experimental scheme of AML mouse model and CD117×CD3 treatment: Day 0, 8-week old NSG mice were sub-lethally irradiated and injected with 4×10⁶HL60-luc-GFP-cKit^{HIGH} cells via the tail vain 3-6 hours later. Day 1, 4×10⁶ human donor T cells were injected i.v., as well as the first dose of 25 μg CD117×CD3. In the therapy group, 25 μg of the bispecific antibody was injected daily for 18 days. Day 21, mice were sacrificed and bone marrow cells were flushed out from their femurs and stained for FACS analysis. (B) Terminal analysis: quantification of GFP⁺ HL60^{HIGH} cells in the bone marrow of mice. In the therapy group, no GFP⁺ cells were present, while in the control groups, engraftment ranged from 0.1-30%. (C) Quantification of human T cells in the bone marrow of mice. The two groups that were injected with human T cells showed T cell engraftment ranging from 0.1-20%. There was no statistically significant difference between the two groups.

FIG. 6: Binding of CD117×CD3 to CD3 was tested by FACS on isolated human T cells.

(A) Amino acid sequence of CD117×CD3. (B) Size exclusion chromatography profile of CD117×CD3. (C) Flow cytometry data showing binding to HL60 c-Kit^{high} cells and human T cells, but not to HL60 wt cells.

FIG. 7: Mass spectrometry of CD117×CD3

(A) Mass spectrometry analysis of CD117×CD3 shows a single peak with a mass of 55526 Dalton. (B) Mass spectrometry analysis of CD117×CD3 after treatment with PnGase reveals N-linked glycosylation of 2057 Dalton. Theoretical mass of CD117×CD3: 53475 Dalton.

FIG. 8: Preparation and quality control of the extracellular portion of c-Kit

(A) Amino acid sequence of domains 4&5 of the extracellular part of human c-Kit. (B) SDS PAGE reveals a single band of recombinant c-Kit. (C) Size exclusion chromatography shows a single major peak at a size of about double the expected size of the recombinant protein, revealing a dimer state.

FIG. 9: Overview of different bispecific antibody formats

Figure has been taken from Spiess et al. 2015 [55], the content of which is incorporated herein by reference.

FIG. 10: Alignment between the scFv sequences of Okt3 (SEQ ID No 27) and its humanized variant, BlinCD3 (SEQ ID NO 28).

Both antibodies share a sequence identity of 93%. The CDRs are marked in bold.

### DETAILED DESCRIPTION OF THE INVENTION

Before the invention is described in detail, it is to be understood that this invention is not limited to the particular component parts of the devices described or process steps of the methods described as such devices and methods may vary. It is also to be understood that the terminology used herein is for purposes of describing particular embodiments only, and is not intended to be limiting. It must be noted that, as used in the specification and the appended claims, the singular forms “a”, “an”, and “the” include singular and/or plural referents unless the context clearly dictates otherwise. It is moreover to be understood that, in case parameter ranges are given which are delimited by numeric values, the ranges are deemed to include these limitation values.

It is further to be understood that embodiments disclosed herein are not meant to be understood as individual embodiments which would not relate to one another. Features discussed with one embodiment are meant to be disclosed also in connection with other embodiments shown herein. If, in one case, a specific feature is not disclosed with one embodiment, but with another, the skilled person would understand that does not necessarily mean that said feature is not meant to be disclosed with said other embodiment. The skilled person would understand that it is the gist of this application to disclose said feature also for the other embodiment, but that just for purposes of clarity and to keep the specification in a manageable volume this has not been done.

Furthermore, the content of the prior art documents referred to herein is incorporated by reference. This refers, particularly, for prior art documents that disclose standard or routine methods. In that case, the incorporation by reference has mainly the purpose to provide sufficient enabling disclosure, and avoid lengthy repetitions.

According to a first aspect of the invention, a bispecific binding agent is provided comprising at least a first binding domain and a second binding domain, wherein said first binding domain binds to CD117/c-KIT and wherein said second binding domain binds to CD3. In another embodiment, said first binding domain binds to CD3 and said second binding domain binds to CD117/c-KIT.

Preferably, CD117/c-KIT is human CD117/c-KIT as e.g. disclosed under UniProt identifier P10721.

In one embodiment, the CD3 binding domain binds to the epsilon-subunit of CD3, preferably human CD3, as e.g. disclosed under UniProt identifier P07766

Mast/stem cell growth factor receptor (SCFR), also known as proto-oncogene c-KIT or tyrosine-protein kinase KIT or CD117, is a receptor tyrosine kinase protein that in humans is encoded by the KIT gene. Multiple transcript variants encoding different isoforms have been found for this gene. KIT was first described by the German biochemist Axel Ullrich in 1987 as the cellular homolog of the feline sarcoma viral oncogene v-kit.

CD117 is a cytokine receptor expressed on the surface of hematopoietic stem cells as well as other cell types. Altered forms of this receptor may be associated with some types of cancer. CD117 is a receptor tyrosine kinase type III, which binds to stem cell factor (a substance that causes certain types of cells to grow), also known as “steel factor” or “c-kit ligand”. When this receptor binds to stem cell factor (SCF) it forms a dimer that activates its intrinsic tyrosine kinase activity, that in turn phosphorylates and activates signal transduction molecules that propagate the signal in the cell.[10] After activation, the receptor is ubiquitinated to mark it for transport to a lysosome and eventual destruction. Signaling through CD117 plays a role in cell survival, proliferation, and differentiation. For instance, CD117 signaling is required for melanocyte survival, and it is also involved in haematopoiesis and gametogenesis.

Like other members of the receptor tyrosine kinase III family, CD117 consists of an extracellular domain, a transmembrane domain, a juxtamembrane domain, and an intracellular tyrosine kinase domain. The extracellular domain is composed of five immunoglobulin-like domains, and the protein kinase domain is interrupted by a hydrophilic insert sequence of about 80 amino acids. The ligand stem cell factor binds via the second and third immunoglobulin domains.

Cluster of differentiation (CD) molecules are markers on the cell surface, as recognized by specific sets of antibodies, used to identify the cell type, stage of differentiation and activity of a cell. CD117 is an important cell surface marker used to identify certain types of hematopoietic (blood) progenitors in the bone marrow. To be specific, hematopoietic stem cells (HSC), multipotent progenitors (MPP), and common myeloid progenitors (CMP) express high levels of CD117. Common lymphoid progenitors (CLP) express low surface levels of CD117. CD117 also identifies the earliest thymocyte progenitors in the thymus—early T lineage progenitors (ETP/DN1) and DN2 thymocytes express high levels of c-Kit. It is also a marker for mouse prostate stem cells. In addition, mast cells, melanocytes in the skin, and interstitial cells of Cajal in the digestive tract express CD117. In humans, expression of c-kit in helper-like innate lymphoid cells (ILCs) which lack the expression of CRTH2 (CD294) is used to mark the ILC3 population.

In humans, CD117/c-Kit is inter alia present in hematopoietic cells and plays a crucial role in early stages of hematopoiesis, but is also expressed in AML, blasts [11-14].

In immunology, the CD3 (cluster of differentiation 3) T cell co-receptor helps to activate both the cytotoxic T cell (CD8+ naive T cells) and also T helper cells (CD4+ naive T cells). It consists of a protein complex and is composed of four distinct chains. In mammals, the complex contains a CD3γ chain, a CD3δ chain, and two CD3ε chains. These chains associate with the T-cell receptor (TCR) and the ζ-chain (zeta-chain) to generate an activation signal in T lymphocytes. The TCR, ζ-chain, and CD3 molecules together constitute the TCR complex.

The CD3γ, CD3δ, and CD3ε chains are highly related cell-surface proteins of the immunoglobulin superfamily containing a single extracellular immunoglobulin domain.

Containing aspartate residues, the transmembrane region of the CD3 chains is negatively charged, a characteristic that allows these chains to associate with the positively charged TCR chains.

The intracellular tails of the CD3 molecules contain a single conserved motif known as an immunoreceptor tyrosine-based activation motif or ITAM for short, which is essential for the signaling capacity of the TCR. The intracellular tails of the ζ chain contain 3 ITAM motifs.

Phosphorylation of the ITAM on CD3 renders the CD3 chain capable of binding an enzyme called ZAP70 (zeta associated protein), a kinase that is important in the signaling cascade of the T cell. Because CD3 is required for T cell activation, drugs (often monoclonal antibodies) that target it are being investigated as immunosuppressant therapies (e.g., otelixizumab (TRX4) for type 1 diabetes and other autoimmune diseases, or Muromonab-CD3 (OKT3®) to reduce acute rejection in patients with organ transplant.

Generally, quite a few multispecific binding agents which target CD3 together with another target are currently in development, or even already approved. One of the first approved bispecific antibodies was Blinatumomab (trade name Blincyto®), which targets CD3 and CD19, and is used as a second-line treatment for Philadelphia chromosome-negative relapsed or refractory acute lymphoblastic leukemia. The basic idea of these constructs is to bind to tumor cells via a tumor specific target, and engage T-cells via the CD3-binding moiety.

So, while said concept seems intriguing, its translation into drugs that offer a clinical advantage is not as easy as it seems. The history of bispecific antibodies is plastered with failures and disappointments.

Catumaxomab, (trade name Removab®) which was approved even earlier than Blinatumomab, and was used for the treatment of malignant ascites in people with EpCAM-positive cancer. It binds CD3 and EpCAM, but was withdrawn from the market in 2017.

The clinical development of Xencor's CD123×CD3 antibody XmAb14045 (WO2017210443) was halted after 2 patient deaths in February 2019. Other products targeting CD19XCD3 (Affimed's AFM11, WO2013013700) and CD3×B7-H3 (Macrogenics' MGD009, U.S. Pat. No. 9,441,049B2) were also withdrawn from the regulatory process, as well as J&J's CD123×CD3 bispecific JNJ-63709178 (EP3189081A1).

As shown in the following, the bispecific binding agent according to the invention (sometimes called CD117×CD3) was capable to potently redirect T cell activity against both HSPCs and AML cells in vitro. In a mouse model of disseminated leukemia, CD117×CD3 completely abrogated AML engraftment into the bone marrow. The product represents an advantageous alternative to anti-c-Kit immunoglobulins for mild preconditioning strategies in patients with various types of hematological conditions, prior to HSCT. Moreover, the potent anti-leukemic activity of CD117×CD3 may contribute to the efficient killing of AML cells, which are notoriously difficult to eradicate. It hence also offers an advantageous alternative to other bispecific antibodies targeting CD3 and a different target.

According to one embodiment, the bispecific binding agent is a mono- or heteromeric protein or peptide or comprises at least one peptide sequence.

According to one embodiment, the bispecific binding agent comprises at least one stretch derived from an antibody.

According to one embodiment, such stretch is at least one complementarity determining region (CDR), preferably in a set of 3 CDRs (CDR1-3) or 6 CDRS (HCDR1-3 and LCDR1-3).

According to one embodiment, such stretch is at least one variable antibody domain, preferably in a set of 2 variable antibody domain (heavy chain variable domain and light chain variable domain)

According to one embodiment of the invention, said first binding domain binds to the extracellular domain of CD117/c-KIT and/or said second binding domain binds to the ε chain of CD3 and/or the γ chain of CD3. According to another embodiment of the invention, said second binding domain binds to the extracellular domain of CD117/c-KIT and/or said first binding domain binds to the ε chain of CD3 and/or the γ chain of CD3.

According to one embodiment of the invention, the bispecific binding agent is in the format of a full-length antibody or an antibody fragment, the latter retaining target binding properties.

Generally, a large number of formats for bispecific antibody has been evaluated. For a review, see, e.g., Spiess et al (2015) [55], the content of which is incorporated herein by reference. Bispecific antibodies are classified into five distinct structural groups:
(i) bispecific IgG (BsIgG)
(ii) IgG appended with an additional antigen-binding moiety
(iii) bispecific antibody fragments
(iv) bispecific fusion proteins and
(v) bispecific antibody conjugates

Further, it is often differentiated between
(i) assymetric bispecific antibodies (heteropolymeric antibodies with different chains comprising binding moieties to different antigens) and
(ii) symmetric bispecific antibodies, (heteropolymeric or monomeric/single chain antibodies, where the different binding moieties to different antigens are on the same chain)

Generally, small formats, which are often in the monomeric/single chain configuration) have a short serum half life, which makes frequent or even constant dosing necessary, yet provides the option to interrupt treatment immediately upon occurrence of side effects, like cytokine storms.

Asymmetric bispecific antibodies often pose problems when it comes to post-translational pairing of the different chains In the simultaneous expression of four different antibody chains (heavy and light chain for each specificity) irregular pairing can occur, leading to 10 different products, out of which only one is the correct variant. These mispairing issues can however be tackled with different technological approaches (see Wang et al, 2018)

See FIG. 10 for an overview of the different formats. Each of these formats brings different properties and advantages and can hence be chosen as a preferred format, inter alia,
binding valency for each antigen,
geometry of antigen-binding sites, c
correctness of chain pairing,
pharmacokinetic half-life
and in some cases effector functions.

According to embodiments of the invention, the bispecific binding agent is in the format of at least one selected from the group consisting of
DVD-Ig
Knobs in Holes
Crossmab
BiTE™
Nanobody
Diabody
DART™
TandAb™′ and/or
scDb-scFv

These formats are well known to the skilled artisan (see Spiess et al (2015) [55] for the definitions). The skilled artisan can use the variable domains and/or sets of CDRs as disclosed herein to create the above mentioned formats by routine measures.

According to one embodiment of the invention, the bispecific binding agent is in the format of a BiTE.

The BiTE format essentially consists of two scFv formatted antibodies joined to one another by means of a peptide linker, usually a linker comprising the amino acid sequence GGGGs (“G4S”). The resulting single chain has a weight of about 55 kD. Said BiTE can have the following formats (with the dash between the variable domains representing optional linkers, and the numbers designating affinity to either CD3 or CD117.)

VH₁-VL₁-VL₂-VH₂
VL₁-VH₁-VH₂-VL₂
VL₁-VH₁-VL₂-VH₂
VH₁-VL₁-VH₂-VL₂

Because of its small size, the BiTE format is associated with a very rapid clearance profile in patients. Blinatumomab, the only clinically-approved BiTE on the market, is administered as 28-day continuous infusion, thanks to the use of an intravenous pump with constant flow. In such way, the opportunity exists to immediately suspend antibody administration if serious adverse events occur.

The scDb-scFv is similar to the BiTE format, only with the difference that the variable domains for the first or second target are duplicated, es e.g. shown in the following:
VH₁-VL₁-VH₁-VL₁-VL₂-VH₂
VL₁-VH₁-VL₁-VH₁-VH₂-VL₂
VL₁-VH₁-VL₁-VH₁-VL₂-VH₂
VH₁-VL₁-VH₁-VL₁-VH₂-VL₂

The TandAb (Tandem Diabody) format is for example as follows:
VL₁-VH₂-VL₂-VH₁
VL₂-VH₁-VL₁-VH₂

The DART format is a dimeric format where two chains are connected to one another by a cysteine linker:
Chain 1: VL₁-VH₂
Chain 2: VH₁-VL₂

According to one embodiment of the invention, the first binding domain binds to CD117/c-KIT
a) comprises a set of heavy chain/light chain complementarity determining regions (CDR) comprised in the heavy chain/light variable region sequence pair of the anti CD117 antibody 79D, as set forth in SEQ ID NOs 19 and 20
b) comprises a set of heavy chain/light chain complementarity determining regions (CDR) comprising the following sequences
   HC CDR1 (SEQ ID NO 1 or 35)
   HC CDR2 (SEQ ID NO 2 or 36)
   HC CDR3 (SEQ ID NO 3 or 37)
   LC CDR1 (SEQ ID NO 4 or 38)
   LC CDR2 (SEQ ID NO 5 or 39), and
   LC CDR3 (SEQ ID NO 6 or 40)
c) comprises the heavy chain/light chain complementarity determining regions (CDR) of b), with the proviso that at least one of the CDRs has up to 3 amino acid substitutions relative to the respective SEQ ID NO 1-6, or SEQ ID NO 35-40, and/or
d) comprises the heavy chain/light chain complementarity determining regions (CDR) of b), with the proviso that at least one of the CDRs has a sequence identity of ≥66% to the respective SEQ ID NO 1-6, or SEQ ID NO 35-40,

wherein the CDRs are embedded in a suitable protein framework so as to be capable to bind to CD117/c-KIT.

The two sets, SEQ ID Nos 1-6 and 35-40, relate to the same antibody, but only to different nomenclatures according to which the CDRs were defined.

As used herein, the term “CDR” or “complementarity determining region” is intended to mean the non-contiguous antigen combining sites found within the variable region of both heavy and light chain polypeptides. These particular regions have been described by Kabat et al. (1977), Kabat et al. (1991), Chothia et al. (1987) and MacCallum et al., (1996) where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison. Note that this numbering may differ from the CDRs that acre actually disclosed in the enclosed sequence listing, because CDR definitions vary from case to case.

TABLE 1
CDR definitions

| | Kabat1 | Chothia2 | MacCallum3 |
|---|---|---|---|
| VH CDR1 | 31-35 | 26-32 | 30-35 |
| VH CDR2 | 50-65 | 53-55 | 47-58 |
| VH CDR3 | 95-102 | 96-101 | 93-101 |
| VL CDR1 | 24-34 | 26-32 | 30-36 |
| VL CDR2 | 50-56 | 50-52 | 46-55 |
| VL CDR3 | 89-97 | 91-96 | 89-96 |

As used herein, the term “framework” when used in reference to an antibody variable region is entered to mean all amino acid residues outside the CDR regions within the variable region of an antibody. Therefore, a variable region framework is between about 100-120 amino acids in length but is intended to reference only those amino acids outside of the CDRs.

In one embodiment, the term “capable to bind to target X” is to be understood as meaning binding with a sufficient binding affinity. In such embodiment, the respective binding domain binds the target with a K_{D} of 10⁻⁴ or smaller. K_{D} is the equilibrium dissociation constant, a ratio of k_{off}/kₒₙ, between the antibody and its antigen. K_{D} and affinity are inversely related. The K_{D} value relates to the concentration of antibody (the amount of antibody needed for a particular experiment) and so the lower the K_{D} value (lower concentration) and thus the higher the affinity of the binding domain. The following table shows typical K_{D} ranges of monoclonal antibodies

TABLE 2
K_{D} and Molar Values

| K_{D} value | Molar range |
|---|---|
| 10⁻⁴ to 10⁻⁶ | Micromolar (μM) |
| 10⁻⁷ to 10⁻⁹ | Nanomolar (nM) |
| 10⁻¹⁰ to 10⁻¹² | Picomolar (pM) |
| 10⁻¹³ to 10⁻¹⁵ | Femtomolar (fM) |

According to one embodiment of the invention, the second binding domain binds to CD3
a) comprises a set of heavy chain/light chain complementarity determining regions (CDR) comprising the following sequences OKT3, BlinCD3
b) comprises a set of heavy chain/light chain complementarity determining regions (CDR) comprised in the heavy chain/light variable region sequence pair of one of the anti CD3 antibodies BlinCD3, OKT3, and [ . . . ], as set forth in SEQ ID NOs 21 and 22, or SEQ ID NO 23 and 24
c) comprises the set of heavy chain/light chain complementarity determining regions (CDR) comprising the following sequences
   HC CDR1 (SEQ ID NO 7 or 41)
   HC CDR2 (SEQ ID NO 8 or 42)
   HC CDR3 (SEQ ID NO 9 or 43)
   LC CDR1 (SEQ ID NO 10 or 44)
   LC CDR2 (SEQ ID NO 11 or 45), and
   LC CDR3 (SEQ ID NO 12 or 46)
d) comprises the set of heavy chain/light chain complementarity determining regions (CDR) comprising the following sequences
   HC CDR1 (SEQ ID NO 13)
   HC CDR2 (SEQ ID NO 14)
   HC CDR3 (SEQ ID NO 15)
   LC CDR1 (SEQ ID NO 16)
   LC CDR2 (SEQ ID NO 17), and
   LC CDR3 (SEQ ID NO 18)
e) comprises the heavy chain/light chain complementarity determining regions (CDR) of b) or c), with the proviso that at least one of the CDRs has up to 3 amino acid substitutions relative to the respective SEQ ID NO 7-12 or SEQ ID NO 41-46 or SEQ ID NO 13-18, and/or
f) comprises the heavy chain/light chain complementarity determining regions (CDR) of b), with the proviso that at least one of the CDRs has a sequence identity of ≥66% to the respective SEQ ID NO 7-12 or SEQ ID NO 41-46 or SEQ ID NO 13-18,

wherein the CDRs are embedded in a suitable protein framework so as to be capable to bind to CD3.

The two sets, SEQ ID Nos 7-12 and 41-46, relate to the same antibody, but only to different nomenclatures according to which the CDRs were defined.

As used herein, 79D is an anti CD117/c-Kit antibody that is described in Reshetnyak et al. 2013, the content of which is incorporated herein by reference.

As used herein, OKT3 is an antibody that is described in Horn et al. 2017, the content of which is incorporated herein by reference.

The INN of OKT3 is muromonab-CD3. OKT3 was approved by the U.S. Food and Drug Administration (FDA) in 1985, making it the first monoclonal antibody to be approved anywhere as a drug for humans. Muromonab-CD3 is a murine antibody provided in the IgG format and targets CD3c. It is approved for the therapy of acute, glucocorticoid-resistant rejection of allogeneic renal, heart and liver transplants. It has also been investigated for use in treating T-cell acute lymphoblastic leukemia.

As used herein, BlinCD3 is an antibody that is described in Drugbank, Accession Number DB09052, AA residues 256-498, the content of which is incorporated herein by reference. BlinCD3 is derived from OKT3 by humanization (Dorken et al, 2018) and has about 93% sequence identity therewith in the variable domains. See FIG. 11 for an alignment, with the CDRs marked in bold. Only LCDRs 2 and 3 differ slightly from one another in the respective C terminal AA residue.

Preferably, the bispecific binding agent has up to 2 amino acid substitutions, and more preferably up to 1 amino acid substitutions

Preferably, at least one of the CDRs has a sequence identity of ≥67%, ≥68%, ≥69%, ≥70%, ≥71%, ≥72%, ≥73%, ≥74%, ≥75%, ≥76%, ≥77%, ≥78%, ≥79%, ≥80%, ≥81%, ≥82%, ≥83%, ≥84%, ≥85%, ≥86%, ≥87%, ≥88%, ≥89%, ≥90%, ≥91%, ≥92%, ≥93%, ≥94%, ≥95%, ≥96%, ≥97%, ≥98%, and most preferably ≥99% to the respective SEQ ID NO.

As used herein, the term “% sequence identity”, has to be understood as follows: Two sequences to be compared are aligned to give a maximum correlation between the sequences. This may include inserting “gaps” in either one or both sequences, to enhance the degree of alignment. A % identity may then be determined over the whole length of each of the sequences being compared (so-called global alignment), that is particularly suitable for sequences of the same or similar length, or over shorter, defined lengths (so-called local alignment), that is more suitable for sequences of unequal length. In the above context, an amino acid sequence having a “sequence identity” of at least, for example, 95% to a query amino acid sequence, is intended to mean that the sequence of the subject amino acid sequence is identical to the query sequence except that the subject amino acid sequence may include up to five amino acid alterations per each 100 amino acids of the query amino acid sequence. In other words, to obtain an amino acid sequence having a sequence of at least 95% identity to a query amino acid sequence, up to 5% (5 of 100) of the amino acid residues in the subject sequence may be inserted or substituted with another amino acid or deleted. Methods for comparing the identity and homology of two or more sequences are well known in the art. The percentage to which two sequences are identical can for example be determined by using a mathematical algorithm. A preferred, but not limiting, example of a mathematical algorithm is integrated in the BLAST family of programs, e.g. BLAST or NBLAST program and FASTA. Sequences which are identical to other sequences to a certain extent can be identified by these programmes. Furthermore, programs available in the Wisconsin Sequence Analysis Package, version 9.1 for example the programs BESTFIT and GAP, may be used to determine the % identity between two polypeptide sequences. If herein reference is made to an amino acid sequence sharing a particular extent of sequence identity to a reference sequence, then said difference in sequence is preferably due to conservative amino acid substitutions. Preferably, such sequence retains the activity of the reference sequence, e.g. albeit maybe at a slower rate.

Preferably, at least one of the CDRs has been subject to CDR sequence modification, including
affinity maturation
reduction of immunogenicity

Affinity maturation in the process by which the affinity of a given antibody is increased in vitro. Like the natural counterpart, in vitro affinity maturation is based on the principles of mutation and selection. It has successfully been used to optimize antibodies, antibody fragments or other peptide molecules like antibody mimetics. Random mutations inside the CDRs are introduced using radiation, chemical mutagens or error-prone PCR. In addition, the genetic diversity can be increased by chain shuffling. Two or three rounds of mutation and selection using display methods like phage display usually results in antibody fragments with affinities in the low nanomolar range. For principles see Eylenstein et al. (2016), the content of which is incorporated herein by reference.

Humanized antibodies contain murine-sequence derived CDR regions that have been engrafted, along with any necessary framework back-mutations, into human sequence-derived V regions. Hence, the CDRs themselves can cause immunogenic reactions when the humanized antibody is administered to a patient. Methods of reducing immunogenicity caused by CDRs are disclosed in Harding et al. (2010), the content of which is incorporated herein by reference.

According to one embodiment of the invention, the framework is a human VH/VL framework. VH stands for heavy chain variable domain of an IgG shaped antibody, while VL stands for light chain variable domain (kappa or lambda)

According to one embodiment of the invention, the first binding domain which binds to CD117/c-KIT comprises

a) the variable domains of of the antibody 79D

b) the heavy chain/light chain variable domains (VD)
HC VD (SEQ ID NO 19), and
LC VD (SEQ ID NO 20)

c) the heavy chain/light chain variable domains (VD) of b), with the proviso that
the HCVD has a sequence identity of ≥80% to the respective SEQ ID NO 19, and/or
the LCDVD has a sequence identity of ≥80% to the respective SEQ ID NO 20,

d) the heavy chain/light chain variable domains (VD) of b), with the proviso that at least one of the HCVD or LCVD has up to 10 amino acid substitutions relative to the respective SEQ ID NO 19 and/or 20,

said bispecific binding agent still being capable to bind to CD117/c-KIT.

A “variable domain” when used in reference to an antibody or a heavy or light chain thereof is intended to mean the portion of an antibody which confers antigen binding onto the molecule and which is not the constant region. The term is intended to include functional fragments thereof which maintain some of all of the binding function of the whole variable region. Variable region binding fragments include, for example, functional fragments such as Fab, F(ab)₂, Fv, single chain Fv (scfv) and the like. Such functional fragments are well known to those skilled in the art. Accordingly, the use of these terms in describing functional fragments of a heteromeric variable region is intended to correspond to the definitions well known to those skilled in the art. Such terms are described in, for example, Huston et al., (1993) or Plückthun and Skerra (1990).

According to one embodiment of the invention, the second binding domain which binds to CD3 comprises
a) the variable domains of one antibody selected from the group consisting of OKT3 and BlinCD3
b) the heavy chain/light chain variable domains (VD)
   HC VD (SEQ ID NO 21 or 23), and
   LC VD (SEQ ID NO 22 or 24)
c) the heavy chain/light chain variable domains (VD) of b), with the proviso that
   the HCVD has a sequence identity of ≥80% to the respective SEQ ID NO 21 or 23, and/or
   the LCDVD has a sequence identity of ≥80% to the respective SEQ ID NO 22 or 24
d) the heavy chain/light chain variable domains (VD) of b), with the proviso that at least one of the HCVD or LCVD has up to 10 amino acid substitutions relative to the respective SEQ ID NOs,

said bispecific binding agent still being capable to bind to CD3

Preferably, at least one of the variable domains has a sequence identity of ≥81%, ≥82%, ≥83%, ≥84%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99 to the respective SEQ ID NO.

According to one embodiment of the invention, at least one amino acid substitution is a conservative amino acid substitution

A “conservative amino acid substitution” has a smaller effect on antibody function than a non-conservative substitution. Although there are many ways to classify amino acids, they are often sorted into six main groups on the basis of their structure and the general chemical characteristics of their R groups.

In one embodiment, a “conservative amino acid substitution” is one in which the amino acid residue is replaced with an amino acid residue having a similar side chain. For example, families of amino acid residues having similar side chains have been defined in the art. These families include amino acids with
basic side chains (e.g., lysine, arginine, histidine),
acidic side chains (e.g., aspartic acid, glutamic acid),
uncharged polar side chains (e.g., glycine, asparagine, glutamine, serine, threonine, tyrosine, cysteine),
nonpolar side chains (e.g., alanine, valine, leucine, isoleucine, proline, phenylalanine, methionine, tryptophan),
beta-branched side chains (e.g., threonine, valine, isoleucine) and
aromatic side chains (e.g., tyrosine, phenylalanine, tryptophan, histidine).

Other conserved amino acid substitutions can also occur across amino acid side chain families, such as when substituting an asparagine for aspartic acid in order to modify the charge of a peptide. Thus, a predicted nonessential amino acid residue in a HR domain polypeptide, for example, is preferably replaced with another amino acid residue from the same side chain family or homologues across families (e.g. asparagine for aspartic acid, glutamine for glutamic acid). Conservative changes can further include substitution of chemically homologous non-natural amino acids (i.e. a synthetic non-natural hydrophobic amino acid in place of leucine, a synthetic non-natural aromatic amino acid in place of tryptophan).

According to embodiments of the invention, the corresponding variable heavy chain regions (VH) and the corresponding variable light chain regions (VL) regions are arranged, from N-terminus to C-terminus, in the order,
V_{L}(CD117)-Linker-V_{H}(CD117)-Linker-V_{H}(CD3)-Linker-V_{L}(CD3),
V_{L}(CD117)-Linker-V_{H}(CD117)-Linker-V_{L}(CD3)-Linker-V_{H}(CD3),
V_{H}(CD117)-Linker-V_{L}(CD117)-Linker-V_{H}(CD3)-Linker-V_{L}(CD3),
V_{H}(CD117)-Linker-V_{L}(CD117)-Linker-V_{L}(CD3)-Linker-V_{H}(CD3),
V_{L}(CD3)-Linker-V_{H}(CD3)-Linker-V_{H}(CD117)-Linker-V_{L}(CD117),
V_{L}(CD3)-Linker-V_{H}(CD3)-Linker-V_{L}(CD117)-Linker-V_{H}(CD117),
V_{H}(CD3)-Linker-V_{L}(CD3)-Linker-V_{H}(CD117)-Linker-V_{L}(CD117), and/or
V_{H}(CD3)-Linker-V_{L}(CD3)-Linker-V_{L}(CD117)-Linker-V_{H}(CD117),

wherein the term “Linker” defines an optional polypeptide linker, as e.g. disclosed in SEQ ID NO 32, 32 or 33. These embodiments all comply with the BiTE format discussed elsewhere herein.

Examples of such embodiments are given in any of SEQ ID Nos 25, 26, 34, 59 and 60. Note that in some sequences a His tag and/or a signal sequence is encompassed. These sequences shall be deemed disclosed with and without His tag and/or a signal sequence.

According to embodiments of the invention, the corresponding variable heavy chain regions (VH) and the corresponding variable light chain regions (VL) regions are arranged, from N-terminus to C-terminus, in the order,
V_{H}(CD117)-Linker-V_{L}(CD117)-Linker-V_{H}(CD117)-Linker-V_{L}(CD117)-Linker V_{X}-(CD3)-Linker-V_{L}(CD3)

This embodiment complies with the scDb-scFv format discussed elsewhere herein. An example of such embodiment is given in SEQ ID NO 61.

According to further embodiments of the invention, the corresponding variable heavy chain regions (VH) and the corresponding variable light chain regions (VL) regions are arranged, from N-terminus to C-terminus, in the order,
V_{L} (CD3)-Linker-V_{H} (CD117)-Linker-V_{L} (CD117)-V_{H} (CD3)

This embodiment complies with the TandAb format discussed elsewhere herein. An example of such embodiment is given in SEQ ID NO 62.

According to further embodiments of the invention, the corresponding variable heavy chain regions (VH) and the corresponding variable light chain regions (VL) regions are arranged in a heterodimeric configuration, from N-terminus to C-terminus, in the order,
Chain 1: V_{L} (CD3)-Linker-V_{H} (CD117)-Cys
Chain 2: V_{L} (CD117)-Linker-V_{H} (CD3)-Cys

with a disulfide bridge established by the two c-terminal cysteine residues. This embodiment complies with the DART format discussed elsewhere herein. An example of such embodiment is given in SEQ ID NOs 63 and 64.

According to one embodiment of the invention, the second binding domain which binds to CD3

a) comprises a scFv-like sequence as set forth in any one of SEQ ID NOs 28 or 29, or

b) comprises an amino acid sequence that has a sequence identity of ≥80% to any one of SEQ ID NOs 28 or 29.

Preferably, the sequence has a has a sequence identity of ≥81%, ≥82%, ≥83%, ≥84%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99 to the respective SEQ ID NO.

According to one embodiment of the invention, the first binding domain which binds to CD117/cKIT

a) comprises a scFv-like sequence as set forth in SEQ ID NO 27, or

b) comprises an amino acid sequence that has a sequence identity of ≥80% to SEQ ID NOs

Preferably, the sequence has a has a sequence identity of ≥81%, ≥82%, ≥83%, ≥84%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99 to the respective SEQ ID NO.

According to one embodiment of the invention, the bispecific binding agent

a) comprises at least one of the sequences selected from SEQ ID NO 25, 26, 34, 59 or 60, or

b) comprises an amino acid sequence that has a sequence identity of ≥80% to SEQ ID NO 25, 26, 34, 59 or 60.

Note that in some sequences a His tag and/or a signal sequence is encompassed. These sequences shall be deemed disclosed with and without His tag and/or a signal sequence.

Likewise, the protection provided by the respective patent claims is meant to encompass embodiments with and without His tag and/or a signal sequence

Preferably, the sequence has a has a sequence identity of ≥81%, ≥82%, ≥83%, ≥84%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99 to the respective SEQ ID NO.

According to one embodiment of the invention, the bispecific binding agent has at least one of
target binding affinity of ≥50% to CD3, and measured by SPR, and/or
target binding affinity of ≥50% to CD117/c-KIT, and measured by SPR,

compared to that of the bispecific binding agent according to claim 14.

As used herein the term “binding affinity” is intended to mean the strength of a binding interaction and therefore includes both the actual binding affinity as well as the apparent binding affinity. The actual binding affinity is a ratio of the association rate over the disassociation rate. Therefore, conferring or optimizing binding affinity includes altering either or both of these components to achieve the desired level of binding affinity. The apparent affinity can include, for example, the avidity of the interaction. For example, a bivalent heteromeric variable region binding fragment can exhibit altered or optimized binding affinity due to its valency.

A suitable method for measuring the affinity of a binding agent is through surface plasmon resonance (SPR). This method is based on the phenomenon which occurs when surface plasmon waves are excited at a metal/liquid interface. Light is directed at, and reflected from, the side of the surface not in contact with sample, and SPR causes a reduction in the reflected light intensity at a specific combination of angle and wavelength. Biomolecular binding events cause changes in the refractive index at the surface layer, which are detected as changes in the SPR signal. The binding event can be either binding association or disassociation between a receptor-ligand pair. The changes in refractive index can be measured essentially instantaneously and therefore allows for determination of the individual components of an affinity constant. More specifically, the method enables accurate measurements of association rates (kₒₙ) and disassociation rates (k_{off}).

Measurements of kₒₙ and k_{off} values can be advantageous because they can identify altered variable regions or optimized variable regions that are therapeutically more efficacious. For example, an altered variable region, or heteromeric binding fragment thereof, can be more efficacious because it has, for example, a higher kₒₙ valued compared to variable regions and heteromeric binding fragments that exhibit similar binding affinity. Increased efficacy is conferred because molecules with higher kₒₙ values can specifically bind and inhibit their target at a faster rate. Similarly, a molecule of the invention can be more efficacious because it exhibits a lower k_{off} value compared to molecules having similar binding affinity. Increased efficacy observed with molecules having lower k_{off} rates can be observed because, once bound, the molecules are slower to dissociate from their target. Although described with reference to the altered variable regions and optimized variable regions of the invention including, heteromeric variable region binding fragments thereof, the methods described above for measuring associating and disassociation rates are applicable to essentially any antibody or fragment thereof for identifying more effective binders for therapeutic or diagnostic purposes.

Methods for measuring the affinity, including association and disassociation rates using surface plasmon resonance are well known in the arts and can be found described in, for example, Jonsson and Malmquist, (1992) and Wu et al. (1998). Moreover, one apparatus well known in the art for measuring binding interactions is a BIAcore 2000 instrument which is commercially available through Pharmacia Biosensor, (Uppsala, Sweden).

Preferably said target binding affinity is ≥51%, ≥52%, ≥53%, ≥54%, ≥55%, ≥56%, ≥57%, ≥58%, ≥59%, ≥60%, ≥61%, ≥62%, ≥63%, ≥64%, ≥65%, ≥66%, ≥67%, ≥68%, ≥69%, ≥70%, ≥71%, ≥72%, ≥73%, ≥74%, ≥75%, ≥76%, ≥77%, ≥78%, ≥79%, ≥80%, ≥81%, ≥82%, ≥83%, ≥84%, ≥85%, ≥86%, ≥87%, ≥88%, ≥89%, ≥90%, ≥91%, ≥92%, ≥93%, ≥94%, ≥95%, ≥96%, ≥97%, ≥98%, and most preferably ≥99% compared to that of the bispecific binding agent.

According to another aspect of the invention, a bispecific binding agent is provided that competes for binding to CD117/c-KIT and CD3 with the bispecific binding agent according to the above description.

As regards the format or structure of such binding agent, the same preferred embodiments as set forth above apply. The term “competes for binding” as used herein, is used in reference to a first binding agent with an activity which binds to the same target as does a second binding agent with an activity, where the second binding agent is a variant of the first binding agent or a related or dissimilar binding agent. The efficiency (e.g., kinetics or thermodynamics) of binding by the first binding agent may be the same as or greater than or less than the efficiency substrate binding by the second binding agent. For example, the equilibrium binding constant (K_{D}) for binding to the substrate may be different for the two binding agents.

Such competition for binding can be suitably measured with a competitive binding assay. Such assays are disclosed in Finco et al. 2011, the content of which is incorporated herein by reference, and their meaning for interpretation of a patent claim is disclosed inn Deng et al 2018, the content of which is incorporated herein by reference.

According to another aspect of the invention, a bispecific binding agent is provided that binds to essentially the same, or the same, epitopes on CD3 and CD117/c-KIT as the bispecific binding agent according to the above description.

In order to test for this characteristic, suitable epitope mapping technologies are available, including, inter alia,
X-ray co-crystallography and cryogenic electron microscopy (cryo-EM)
Array-based oligo-peptide scanning
Site-directed mutagenesis mapping
High-throughput shotgun mutagenesis epitope mapping
Hydrogen-deuterium exchange
Cross-linking-coupled mass spectrometry

These methods are, inter alia, disclosed and discussed in Banik et al (2010), and DeLisser (1999), the content of which is herein incorporated by reference.

According to another aspect of the invention, a nucleic acid is provided that encodes for a binding agent according to any one of the aforementioned claims.

A given sequence of the encoded binding agent provided, such nucleic acid can have different sequences due to the degeneracy of the genetic code.

Such nucleic acid can be used for pharmaceutic purposes. In such case, it is an RNA-derived molecule that is administered to a patient, wherein the protein expression machinery of the patient expresses the respective binding agent. The mRNA can for example be delivered in suitable liposomes and comprises either specific sequences or modified uridine nucleosides to avoid immune responses and/or improve folding and translation efficiency, sometimes comprising cap modifications at the 5′- and/or 3′ terminus to target them to specific cell types.

Such nucleic acid can be used for transfecting an expression host to then express the actual binding agent. In such case, the molecule can be a cDNA that is optionally integrated into a suitable vector.

According to another aspect of the invention, a pharmaceutical composition is provided which comprises a bispecific binding agent or a nucleic acid according to any one of the aforementioned claims, and optionally one or more pharmaceutically acceptable excipients.

According to another aspect of the invention, a combination is provided which comprises (i) the bispecific binding agent or pharmaceutical composition according to any one of the aforementioned claims and (ii) one or more therapeutically active compounds.

According to another aspect of the invention, the bispecific binding agent, pharmaceutical composition or combination according to any one of the aforementioned claims is provided (for the manufacture of a medicament) in the treatment of a human or animal subject
being diagnosed for,
suffering from or
being at risk of

developing a neoplastic disease, or for the prevention of such condition.

According to another aspect of the invention, a method is provided for treating or preventing a neoplastic disease, comprising administering to a subject in need thereof an effective amount of the bispecific binding agent, pharmaceutical composition or combination according to any one of the aforementioned claims

Preferably, the neoplastic disease is selected from the group consisting of Acute Myeloid Leukemia (AML) and Myelodysplastic Syndrome (MDS).

According to another aspect of the invention, a therapeutic kit of parts is provided, which comprises
a) the bispecific binding agent, pharmaceutical composition or combination according to any one of the aforementioned claims
b) an apparatus for administering the composition, composition or combination, and
c) instructions for use.

Such embodiment is, for example, a pen or syringe for self administration by the patient, or a plaster for transdermal application.

### EXAMPLES

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

All amino acid sequences disclosed herein are shown from N-terminus to C-terminus; all nucleic acid sequences disclosed herein are shown 5′->3′.

Materials and Methods

1. Construction of Bispecific Antibody

The bispecific antibody was constructed from the sequences of the anti-human c-Kit antibody 79D [19], (scFv sequence shown as SEQ ID NO 29 herein), and a humanized version of the anti-human CD3 antibody OKT3 [20], called BlinCD3 (scFv sequence shown as SEQ ID NO 28 herein). It was genetically assembled by successive overlap PCR in the order VL_{79D}-Linker₁₅ₐₐ-VH_{79D}-Linker₁₅ₐₐ-VH_{BlinCD3}-Linker₁₈ₐₐ-VL_{BlinCD3}-His₆ and cloned into the pcDNA3.1 expression vector downstream of a mammalian excretion signal sequence. In the first step, cDNA sequences of VL_{79D}, VH_{79D}, and VH_{BlinCD3} were amplified. In the second step, VL_{79D}-Linker₁₅ₐₐ-VH_{79D} and VH_{BlinCD3}-Linker₁₈ₐₐ-VL_{BlinCD3} fragments were assembled and in a final cloning step the two scFv-coding fragments were assembled to yield the full construct. The final fragment was then digested with HindIII-HF and NotI according to the manufacturers protocol and ligated into the expression vector.

2. Cell Lines

For the production of the bispecific antibody, Chinese hamster ovary cells (CHO-S, Invitrogen) in suspension were used. CHO-S cells in suspension were cultured in shaker incubators (37° C.) in PowerCHO medium, supplemented with 8 mM Ultraglutamin and HT supplement.

HL-60 cells (CCL-240™, ATCC®, Manassas, Va., USA) were cultured in Dulbeccos' Modified Eagle Medium (DMEM) supplemented with 10% FCS and 1% penicillin/streptomycin.

3. Protein Expression and Purification

The bispecific antibody was produced by transient gene expression. For 11 of production, 4×10⁹ CHO-S cells were resuspended in PROCHO4 (Lonza). Plasmid DNA (3200 μg) and 10 ml 1 mg/ml polyethylene imine (PEI) was added to the cell suspension. The transfected cultures were then incubated in a shaker incubator at 31° C. for 6 days. The fusion proteins were purified from the cell culture medium by protein A affinity chromatography and then dialyzed against phosphate buffered saline (PBS).

4. Surface Plasmon Resonance

Bispecific antibody solutions were filtered (using 0.22 μm PVDF filters) and their binding properties analyzed using a BTAcore S200 instrument and an antigen coated CM5 sensor chip (GE Healthcare), which was prepared by covalent coupling of human c-Kit domains 4&5 according to the manufacturer's protocol.

5. Isolation of T-Cells

Human PBMCs were enriched by density gradient centrifugation (Ficoll-Paque Plus, GE Healthcare, Chicago, Ill., USA) from excess buffy coats of healthy donors (provided by Blutspendedienst Zurich, Switzerland). Human T cells were negatively isolated using EasySep™ beads (human T cell isolation kit, STEMCELL Technologies, Vancouver, Canada). Purified T cells were cryopreserved in 90% FCS supplemented with 10% DMSO and stored in liquid nitrogen until thawing.

6. AML Patient Cells & Bone Marrow Cells from Healthy Donors

Healthy bone marrow donors and AML patients gave written informed consent and studies were approved by the ethics board of the canton Zurich, Switzerland (2009-0062). Peripheral blood samples of AML patients in remission were collected at the department of Medical Oncology and Hematology, University Hospital Zurich (Zurich, Switzerland). All bone marrow derived human cells were processed by the biobank of the department of Medical Oncology and Hematology, University Hospital Zurich. Cell collection and cryo-preservation procedures were described before. Bone marrow aspirates were acquired from AML patients during initial diagnostic work-up. Mononucleated cells (MNC) were enriched by density gradient centrifugation, before CD3⁺ and CD19⁺ cells were depleted on LD columns by bead-based immunomagnetic selection (both Miltenyi Biotech). BM derived healthy HSPC were isolated from leftover transfusion bags and infusion systems used for allogeneic BM transplantation. Following enrichment of MNC by density gradient centrifugation, CD34⁺ cells were purified by CD34 microbeads and LS columns (both Milltenyi Biotech). All primary human cells were cryo-preserved in freezing media as described before.

7. In Vitro Cytotoxicity Assays

A flow cytometric cytotoxicity assay was adapted from previously published protocols and used for quantification of T-cell activity against AML cell lines, primary bone marrow cells and AML patient samples. In short, AML cell lines (CD117^{negative}, CD117^{low}, CD117^{med} or CD117^{high}) were stained with the PKH26 Red Fluorescent Cell Linker Kit for General Cell Membrane Labeling (Sigma Aldrich), according to the manufacturer's protocol. Target cells were mixed with human T cells at an effector-to-target ratio of 10:1 in advanced RPMI, supplemented with 10% FCS, 2 mM Glutamax™ and penicillin/streptomycin (100 U/ml/100 μg/ml, all Gibco®, Thermo Fisher Scientific, Waltham, Mass., USA) and incubated for 24 h. Prior to cytotoxicity assay initiation, CAR T-cells were cultured in medium without IL-2 overnight. Bispecific antibody was diluted in medium and added in the specified concentrations. After 24 h, cells were stained with TO-PRO™-3 Iodide (Thermo Fisher) and analysed by flow cytometry. Specific killing was calculated by the formula: [1−live target cells (sample)/live target cells (control)]·100, as described before [21].

To quantify specific lysis of hematopoietic cells from healthy bone marrow (BM) donors, total BM-derived mononucleated cells were co-cultured with T-cells at an effector-to-target ratio of 10:1 for 48 h. Co-culture was initiated immediately after thawing.

For analysis of specific lysis against primary human AML cells, autologous human T-cells were co-cultured with BM-derived mononucleated cells from AML patients at an effector-to-target ratio of 10:1 for 48 hours. Before initiation of co-culture, mononucleated cells were thawed and cultured for 3 days in IMDM supplemented with 20% FCS, 1% penicillin/streptomycin, Glutamax™, 50 μM 2 mercaptoethanol, 10 ng/ml stem cell factor (SCF), 50 ng/ml thrombopoietin (TPO), 10 ng/ml IL-6 and 10 ng/ml Flt3 ligand (all recombinant human proteins, Peprotech®, Rocky Hill, N.J., USA).

Cells were stained for expression of CD3, CD117, CD45 and LIVE/DEAD™ Fixable Aqua Dead Cell Stain Kit and analyzed by flow cytometry as described above.

8. BiTE Treatment of AML Mice

A schematic representation of the experimental design is shown in FIG. 5A. In short, NSG mice were sub-lethally irradiated and subsequently injected with 4*10⁶ HL60-Luc-GFP cells intravenously. The day after, 4*10⁶ human T cells were injected i.v., yielding an effector to target ratio of 1:1. Starting on day 1, mice in the treatment group were injected with 25 CD117×CD3 daily for 18 days. On day 21, mice were sacrificed. Their femurs were flushed with PBS and bone marrow cells were further processed for FACS analysis.

9. Flow Cytometry

Single cell suspensions were prepared from mouse bone marrow. Red blood cells were lysed with RBC lysis buffer (BioLegend, San Diego, Calif., USA). Zombie Aqua (BioLegend) was used for exclusion of dead cells. Surface antigens were stained with fluorescently labelled antibodies. Data was acquired on BD FACSCanto or BD LSRFortessa II flow cytometers (both Becton Dickinson, Franklin Lakes, N.J., USA). FlowJo (v10.0.7, FlowJo, LLC, Ashland, Oreg., USA) was used for data analysis and presentation.

Results

1. Cloning, Expression and Characterization of CD117×CD3

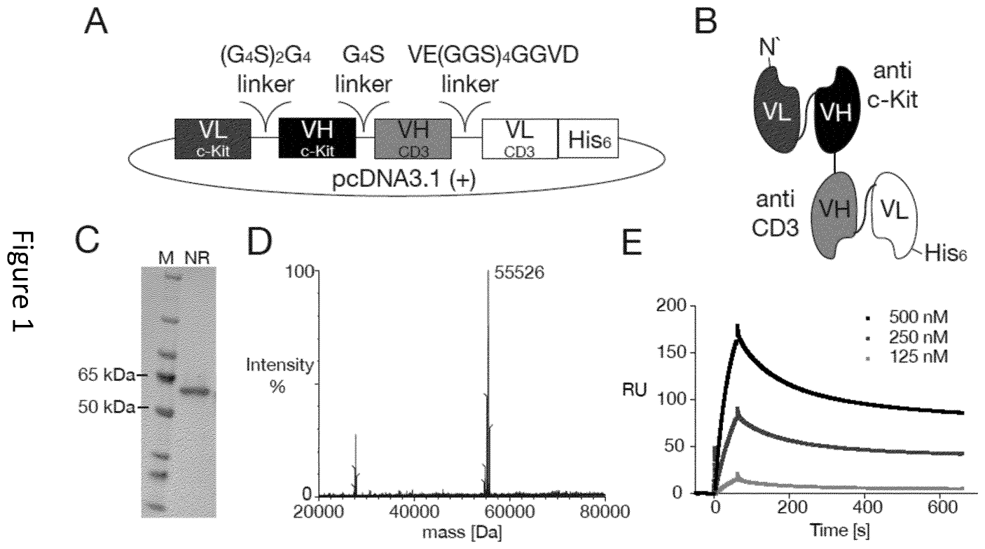

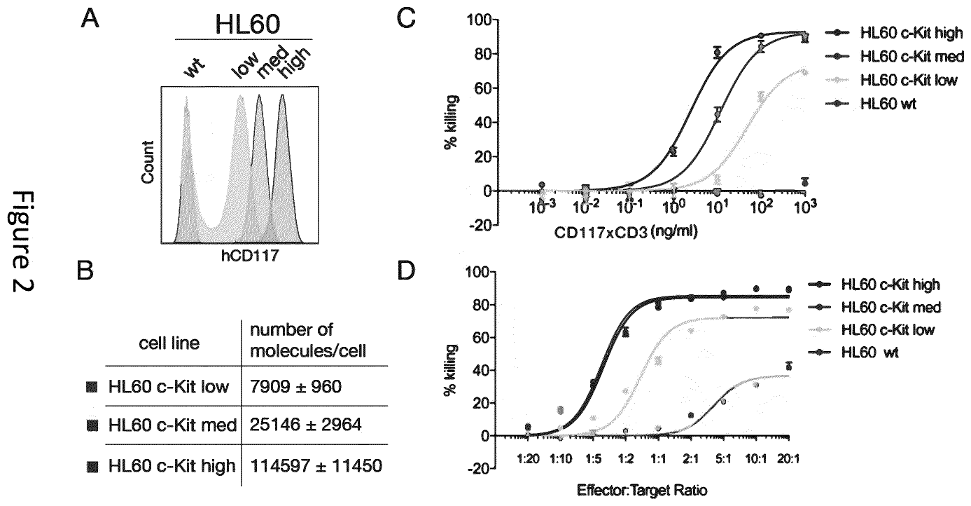

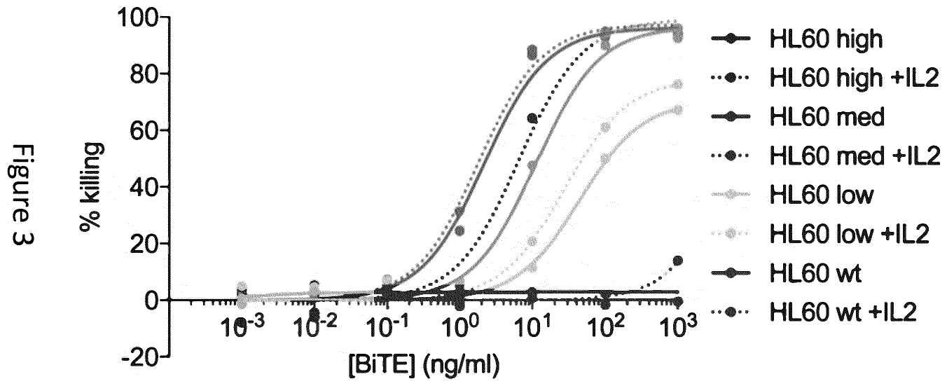

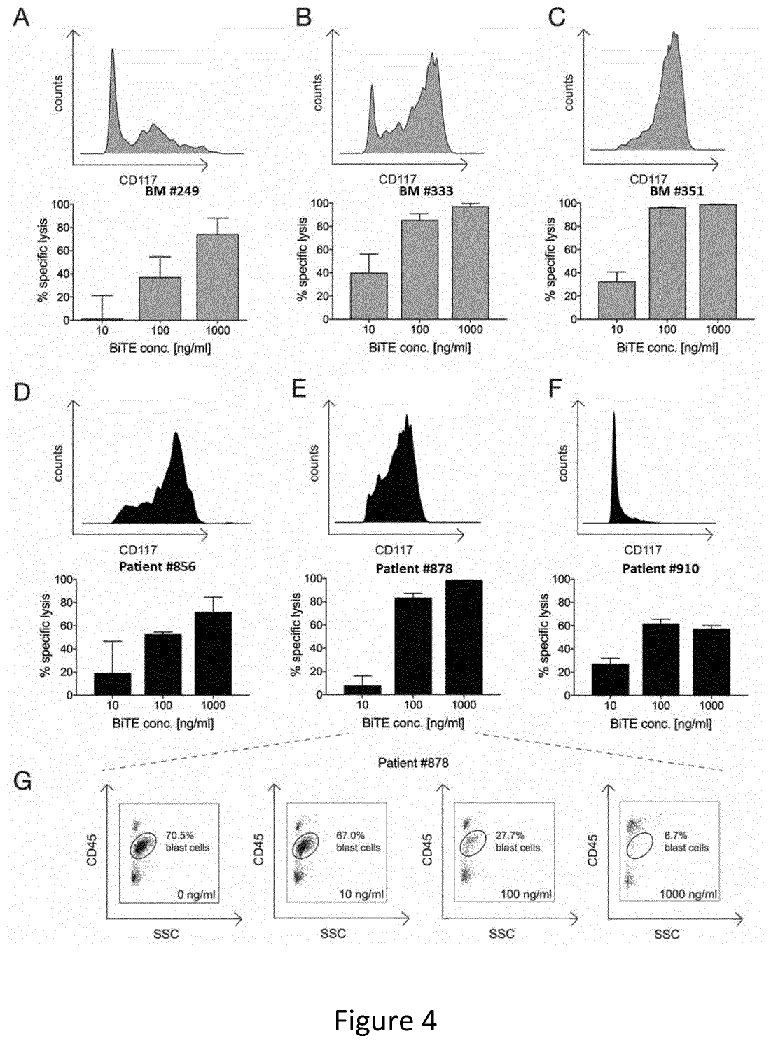

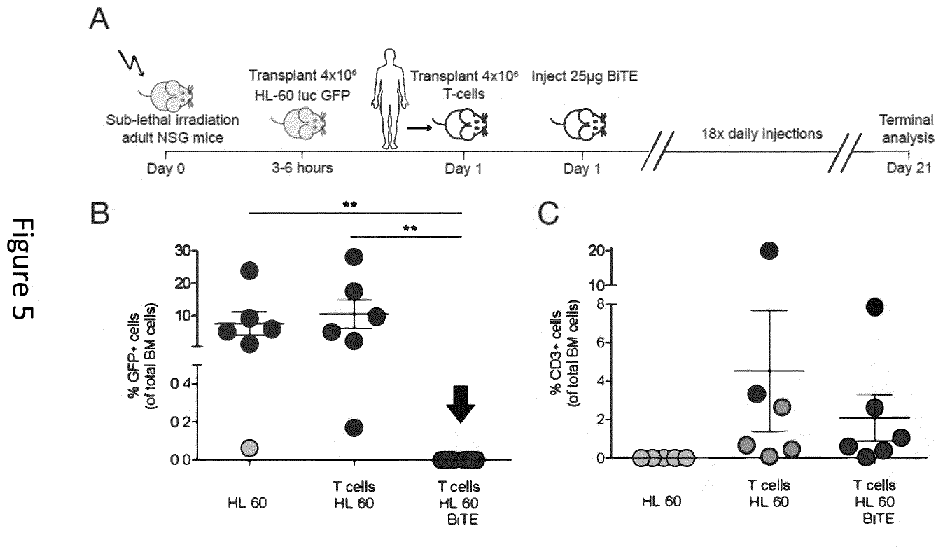

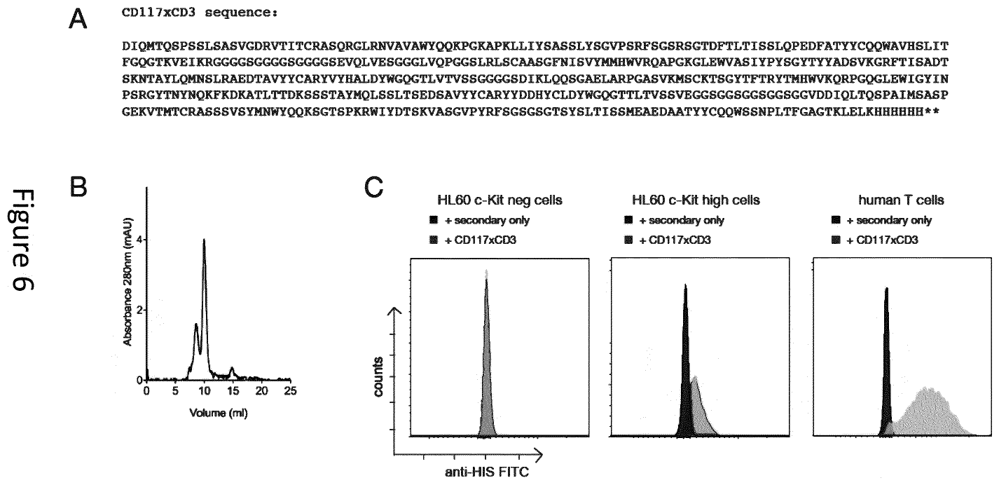

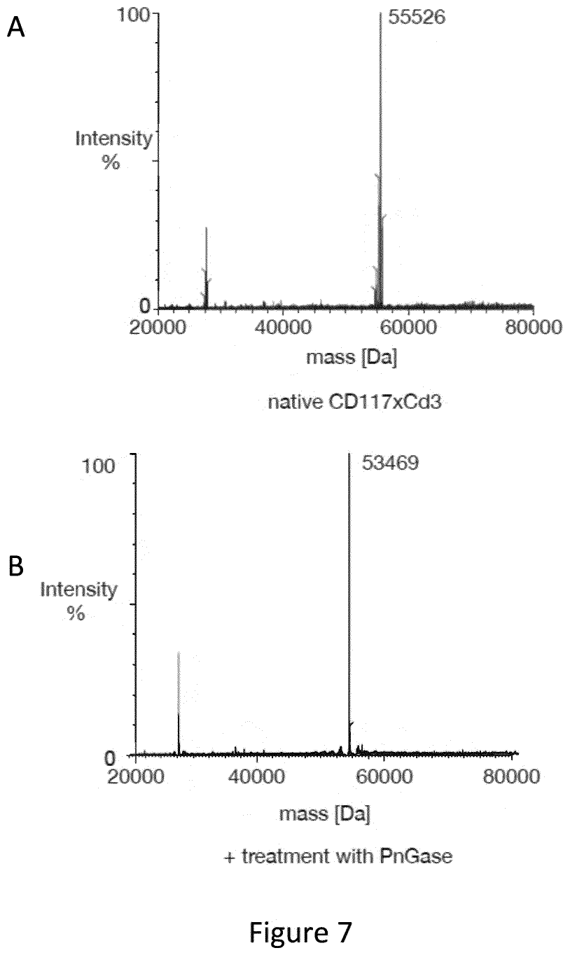

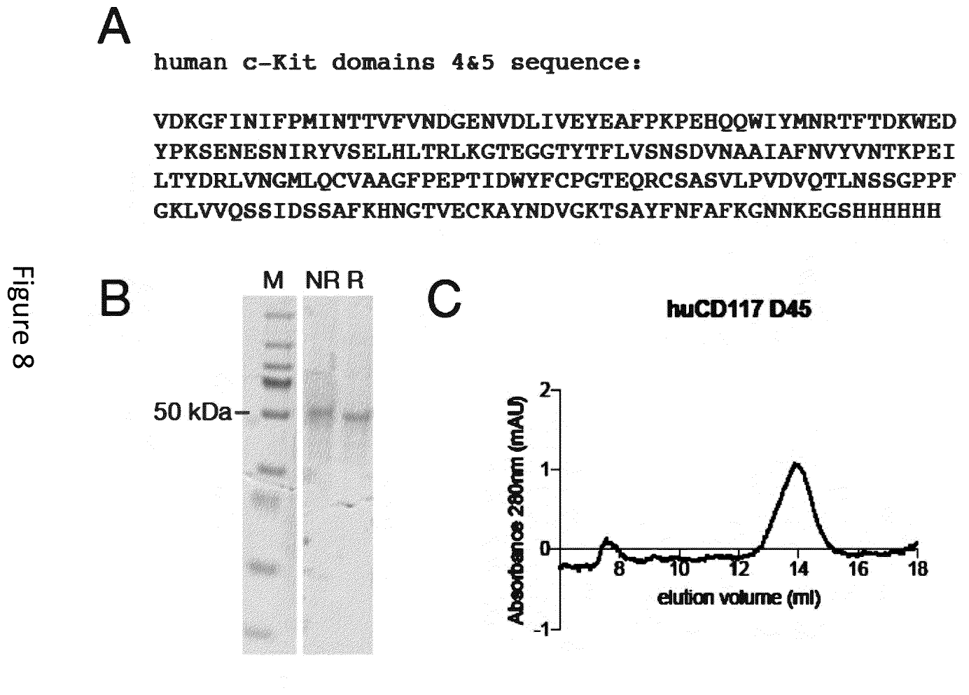

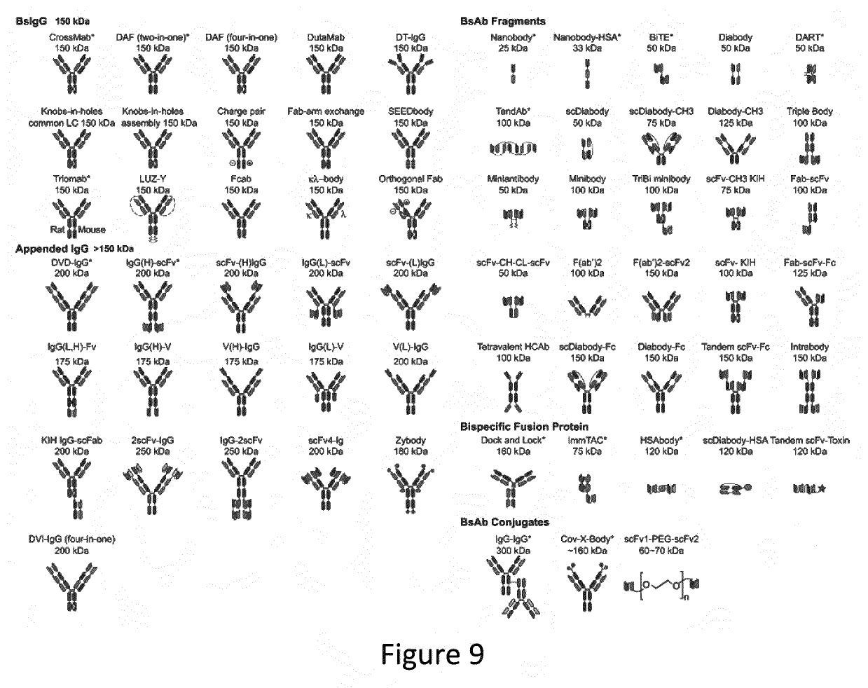

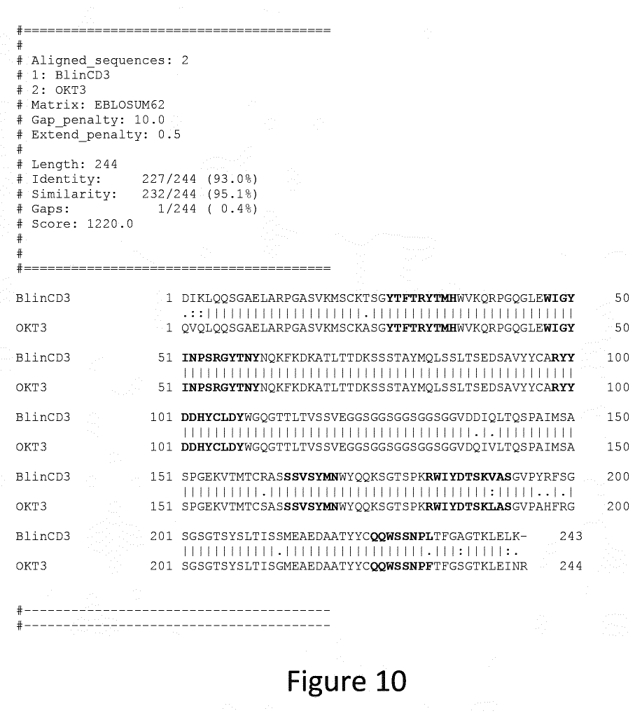

CD117×CD3 was cloned and expressed in BiTE™ format, choosing a VL-VH-VH-VL arrangement for the variable antibody domains [FIG. 1A,B]. We used the 79D antibody for c-Kit recognition [19], while the OKT3 moiety was chosen for CD3 targeting, as this scFv fragment has been successfully used within the blinatumomab structure [22]. A short Gly4Ser linker was selected as linker between the two scFv fragments composing the BiTE™ product, in order to minimize undesired pairing between variable antibody domains or Tandab formation [23]. The fusion protein was cloned into a pcDNA3.1-derived vector (Invitrogen) and expressed in CHO cells by transient gene expression methodologies [24]. The full amino acid sequence of the product and additional protein characterization can be found in FIG. 2. CD117×CD3 was purified to homogeneity using Protein A chromatography, as the 79D antibody V_{H} domain belongs to the VH₃ family and is capable of Protein A binding [25]. A single band was visible by SDS-PAGE electrophoresis [FIG. 1C]. Mass spectrometric analysis revealed a single peak [FIG. 1D], whose mass (55′528 Da) was however larger than the predicted one (53′475 Da), as a result of N-linked glycosylation [FIG. 7].

Preparation and quality control of the extracellular portion of c-Kit is described in FIG. 8, since this protein facilitated the functional characterization of CD117×CD3. The binding properties to recombinant c-Kit were tested by BIAcore analysis, revealing kinetic association and dissociation profiles which were similar to the ones previously reported for the 79D antibody in scFv format [FIG. 1D][19]. Binding of CD117×CD3 to CD3 on human T cells was tested by FACS on isolated human T cells [FIG. 6]

2. Biological Activity of CD117×CD3

In order to characterize the biocidal properties of CD117×CD3, we generated AML cell lines, derived from the HL60 parental line [26], which display different levels of c-Kit on their surface. We focused our attention on three sub-lines (termed HL60 c-Kit high, med and low), which express approximately 8′000, 25′000 and 115′000 c-Kit copies per cell, respectively, as measured using FACS methodologies [FIG. 2A,B]. A dose-dependent in vitro killing of the three cell lines could be observed in the presence of various concentrations of CD117×CD3 and of human T cells at an Effector:Target cell ratio of 10:1 [FIG. 2C]. The HL60 c-Kit high cell line could be efficiently eradicated at 100 ng/ml of BiTE™, corresponding to ˜1.8 nM. We repeated similar experiments using CD117×CD3 at 1000 ng/ml and different Effector:Target cell ratios. We observed that both HL60 c-Kit high and c-Kit med cells could be quantitatively killed at Effector:Target ratios as low as 1:1, while the removal of c-Kit low cells required a higher density of human T cells [FIG. 2D]. The biocidal effect on non-transduced HL60 cells was negligibly low, confirming the requirement of a simultaneous engagement of c-Kit and CD3 antigen for target cell killing.

We have recently reported that the antibody-based targeted delivery of interleukin-2 (IL2) to the bone marrow or to chloroma lesions of AML patients may be associated with a potent anti-leukemic activity [27, 28]. For this reason, we performed cell killing experiments in the presence and in the absence of recombinant human IL2. In this in vitro setting, however, addition of IL2 did not change the cell killing properties of CD117×CD3 [FIG. 3].

In order to characterize the biocidal activity of CD117×CD3 in a setting closer to the clinical situation we performed in vitro cell killing experiments using HSPCs from healthy donors [FIG. 4A,B] and patient-derived AML, blasts [FIG. 4C,D]. CD117×CD3 could efficiently kill both types of target cells in a concentration dependent manner. Complete cell lysis at 1 μg/ml CD117×CD3 concentration was observed for those specimens, characterized by an intense CD117 staining by FACS (i.e., ⅔ HSPC samples and for ⅓ samples of AML blasts) [FIG. 4]. The two-dimensional FACS profiles of FIG. 4E illustrate one example of selective and dose-dependent AML blast killing by the CD117×CD3 BiTE™ in the presence of autologous T cells.

We then explored the anti-leukemic properties of CD117×CD3 in an in vivo system. Sub-lethally irradiated NSG mice received an intravenous injection of 4×10⁶ luciferase- and GFP-transduced HL60 c-Kit high cells, followed by administration of 4×10⁶ human T cells and repeated injections of CD117×CD3 (25 μg/day for 18 days) [FIG. 5A]. Intraperitoneal injection of Luciferin allowed to monitor disease progression by IVIS imaging [29] [FIG. 9]. Mice were euthanized at day 21 and the presence of leukemic stem cells in the bone marrow was assessed by FACS. FIG. 5B shows that AML engraftment into the bone marrow was observed in all control mice, receiving either saline injection or T cells only. By contrast, the metronomic administration of CD117×CD3 led to a complete inhibition of AML engraftment. No statistically-significant difference in human T cell density in the bone marrow could be observed between mice receiving T cells in the presence of saline or in the CD117×CD3 treatment group [FIG. 5C], suggesting that the T cells had not been expanded by day 21, as a result of BiTE™ treatment.

Discussion

We have described novel bispecific antibodies, which bind to human c-Kit (expressed on HSCPs and AML blast cells) and to the CD3 antigen (expressed on T cells). The antibodies are able, inter alia, to selectively kill AML cell lines expressing various levels of c-Kit on their surface in a concentration- and antigen-density dependent manner, in the presence of T cells. The bispecific antibodies also lysed bone marrow cells from healthy donors, which are positive for c-Kit. In vivo, the antibodies completely prevented the engraftment of a c-Kit-positive cell line into the bone marrow of mice.

### REFERENCES

1. Orkin, S. H. and L. I. Zon, Hematopoiesis: an evolving paradigm for stem cell biology. Cell, 2008. 132(4): p. 631-44.
2. Ng, S. W., et al., A 17-gene stemness score for rapid determination of risk in acute leukaemia. Nature, 2016. 540(7633): p. 433-437.
3. Bonnet, D. and J. E. Dick, Human acute myeloid leukemia is organized as a hierarchy that originates from a primitive hematopoietic cell. Nat Med, 1997. 3(7): p. 730-7.
4. Gyurkocza, B. and B. M. Sandmaier, Conditioning regimens for hematopoietic cell transplantation: one size does not fit all. Blood, 2014. 124(3): p. 344-53.
5. Dohner, H., et al., Diagnosis and management of AML in adults: 2017 ELN recommendations from an international expert panel. Blood, 2017. 129(4): p. 424-447.
6. Buckley, S. A., et al., Prediction of adverse events during intensive induction chemotherapy for acute myeloid leukemia or high-grade myelodysplastic syndromes. Am J Hematol, 2014. 89(4): p. 423-8.
7. Chhabra, A., et al., Hematopoietic stem cell transplantation in immunocompetent hosts without radiation or chemotherapy. Sci Transl Med, 2016. 8(351): p. 351ra105.
8. Majeti, R., et al., CD47 is an adverse prognostic factor and therapeutic antibody target on human acute myeloid leukemia stem cells. Cell, 2009. 138(2): p. 286-99.
9. Czechowicz, A., et al., Efficient transplantation via antibody-based clearance of hematopoietic stem cell niches. Science, 2007. 318(5854): p. 1296-9.
10. AMG191, Conditioning/CD34+CD90 Stem Cell Transplant Study for SCID Patients:. 2016.
11. Lennartsson, J. and L. Ronnstrand, Stem cell factor receptor/c-Kit: from basic science to clinical implications. Physiol Rev, 2012. 92(4): p. 1619-49.
12. Tsao, A. S., et al., C-kit receptor expression in acute leukemias-association with patient and disease characteristics and with outcome. Leuk Res, 2004. 28(4): p. 373-8.
13. Valverde, L. R., et al., C-kit receptor (CD117) expression in acute leukemia. Ann Hematol, 1996. 72(1): p. 11-5.
14. Wells, S. J., et al., CD117/CD34 expression in leukemic blasts. Am J Clin Pathol, 1996. 106(2): p. 192-5.
15. Schanzer, J. M., et al., A novel glycoengineered bispecific antibody format for targeted inhibition of epidermal growth factor receptor (EGFR) and insulin-like growth factor receptor type I (IGF-1R) demonstrating unique molecular properties. J Biol Chem, 2014. 289(27): p. 18693-706.
16. Goebeler, M. E. and R. Bargou, Blinatumomab: a CD19/CD3 bispecific T cell engager (BiTE) with unique anti-tumor efficacy. Leuk Lymphoma, 2016. 57(5): p. 1021-32.
17. Neri, D., et al., High-affinity antigen binding by chelating recombinant antibodies (CRAbs). J Mol Biol, 1995. 246(3): p. 367-73.
18. Nagorsen, D., et al., Blinatumomab: a historical perspective. Pharmacol Ther, 2012. 136(3): p. 334-42.
19. Reshetnyak, A. V., et al., Structural basis for KIT receptor tyrosine kinase inhibition by antibodies targeting the D4 membrane-proximal region. Proc Natl Acad Sci USA, 2013. 110(44): p. 17832-7.
20. Horn, L. A., et al., CD3×PDL1 bi-specific T cell engager (BiTE) simultaneously activates T cells and NKT cells, kills PDL1(+) tumor cells, and extends the survival of tumor-bearing humanized mice. Oncotarget, 2017. 8(35): p. 57964-57980.
21. Schlereth, B., et al., Potent inhibition of local and disseminated tumor growth in immunocompetent mouse models by a bispecific antibody construct specific for Murine CD3. Cancer Immunol Immunother, 2006. 55(7): p. 785-96.
22. Loffler, A., et al., A recombinant bispecific single-chain antibody, CD19×CD3, induces rapid and high lymphoma-directed cytotoxicity by unstimulated T lymphocytes. Blood, 2000. 95(6): p. 2098-103.
23. Yang, F., W. Wen, and W. Qin, Bispecific Antibodies as a Development Platform for New Concepts and Treatment Strategies. Int J Mol Sci, 2016. 18(1).
24. Derouazi, M., et al., Serum-free large-scale transient transfection of CHO cells. Biotechnol Bioeng, 2004. 87(4): p. 537-45.
25. Hoogenboom, H. R. and G. Winter, By-passing immunisation. Human antibodies from synthetic repertoires of germline VH gene segments rearranged in vitro. J Mol Biol, 1992. 227(2): p. 381-8.
26. Birnie, G. D., The HL60 cell line: a model system for studying human myeloid cell differentiation. Br J Cancer Suppl, 1988. 9: p. 41-5.
27. Gutbrodt, K. L., et al., Antibody-based delivery of interleukin-2 to neovasculature has potent activity against acute myeloid leukemia. Sci Transl Med, 2013. 5(201): p. 201ra118.
28. Schliemann, C., et al., Targeting interleukin-2 to the bone marrow stroma for therapy of acute myeloid leukemia relapsing after allogeneic hematopoietic stem cell transplantation. Cancer Immunol Res, 2015. 3(5): p. 547-56.
29. Zinn, K. R., et al., Noninvasive bioluminescence imaging in small animals. ILAR J, 2008. 49(1): p. 103-15.
30. Czechowicz, A., et al., Selective hematopoietic stem cell ablation using CD117-antibody-drug-conjugates enables safe and effective transplantation with immunity preservation. Nat Commun, 2019. 10(1): p. 617.
31. Kontermann, R. E., Dual targeting strategies with bispecific antibodies. MAbs, 2012. 4(2): p. 182-97.
32. Portell, C. A., C. M. Wenzell, and A. S. Advani, Clinical and pharmacologic aspects of blinatumomab in the treatment of B-cell acute lymphoblastic leukemia. Clin Pharmacol, 2013. 5(Suppl 1): p. 5-11.
33. Brinkmann, U. and R. E. Kontermann, The making of bispecific antibodies. MAbs, 2017. 9(2): p. 182-212.
34. Rodney A. Prell, D. W. L., Wendy G. Halpern, Anu V. Connor, Chapter 14—Nonclinical Development of Multi-targeting Biopharmaceuticals. Nonclinical Development of Novel Biologics, Biosimilars, Vaccines and Specialty Biologics, ed. D. J. H. Lisa M. Plitnick. 2013: Academic Press.
35. Yuraszeck, T., S. Kasichayanula, and J. E. Benjamin, Translation and Clinical Development of Bispecific T-cell Engaging Antibodies for Cancer Treatment. Clin Pharmacol Ther, 2017. 101(5): p. 634-645.
36. Hipp, S., et al., A novel BCMA/CD3 bispecific T-cell engager for the treatment of multiple myeloma induces selective lysis in vitro and in vivo. Leukemia, 2017. 31(8): p. 1743-1751.
37. Friedrich, M., et al., Regression of human prostate cancer xenografts in mice by AMG 212/BAY2010112, a novel PSMA/CD3-Bispecific BiTE antibody cross-reactive with non-human primate antigens. Mol Cancer Ther, 2012. 11(12): p. 2664-73.
38. Smith, E. J., et al., A novel, native-format bispecific antibody triggering T-cell killing of B-cells is robustly active in mouse tumor models and cynomolgus monkeys. Sci Rep, 2015. 5: p. 17943.
39. Kabat et al., J. Biol. Chem. 252, 6609-6616 (1977) and Kabat et al., Sequences of protein of immunological interest. (1991)
40. Chothia et al., J. Mol. Biol. 196:901-917 (1987)
41. MacCallum et al., J. Mol. Biol. 262:732-745 (1996)
42. Reshetnyak, A. V., et al., Structural basis for KIT receptor tyrosine kinase inhibition by antibodies targeting the D4 membrane-proximal region. Proc Natl Acad Sci USA, 2013. 110(44): p. 17832-7.
43. Horn, L. A., et al., CD3×PDL1 bi-specific T cell engager (BiTE) simultaneously activates T cells and NKT cells, kills PDL1(+) tumor cells, and extends the survival of tumor-bearing humanized mice. Oncotarget, 2017. 8(35): p. 57964-57980.
44. Dorken B, Riethmuller G, Kufer P, Lutterbuse R, Bargou R, Loffler A. inventors; Micromet Ag, assignee. CD19×CD3 specific polypeptides and uses thereof. United States patent U.S. Pat. No. 7,575,923 B2. 2009. Aug. 18
45. Eylenstein, et al, Molecular basis of in vitro affinity maturation and functional evolution of a neutralizing anti-human GM-CSF antibody, mAbs, 8:1, 176-186 (2016)
46. Harding, The immunogenicity of humanized and fully human antibodies. MAbs. 2010 May-June; 2(3): 256-265.
47. Huston et al., Cell Biophysics, 22:189-224 (1993);
48. Plückthun and Skerra, Meth. Enzymol., 178:497-515 (1989) and in Day, E. D., Advanced Immunochemistry, Second Ed., Wiley-Liss, Inc., New York, N.Y. (1990)
49. Jonsson and Malmquist, Advances in Biosensors, 2:291-336 (1992)
50. Wu et al. Proc. Natl. Acad. Sci. USA, 95:6037-6042 (1998).
51. Finco et al, Comparison of competitive ligand-binding assay and bioassay formats for the measurement of neutralizing antibodies to protein therapeutics. J Pharm Biomed Anal. 2011 Jan. 25; 54(2):351-8. doi: 10.1016/j.jpba.2010.08.029. Epub 2010 Sep. 21
52. Deng et al., Enhancing antibody patent protection using epitope mapping information MAbs. 2018 February-March; 10(2): 204-209.
53. Banik, S S R; Doranz, B J (2010). “Mapping complex antibody epitopes”. Genetic Engineering & Biotechnology News. 3 (2): 25-8.
54. DeLisser, H M (1999). Epitope mapping. Methods Mol Biol. 96. pp. 11-20.
55. Spiess C et al, Alternative molecular formats and therapeutic applications for bispecific antibodies. Mol Immunol. 2015 October; 67(2 Pt A):95-106.
56. Wang, A systematic approach for analysis and characterization of mispairing in bispecific antibodies with asymmetric architecture. MAbs. 2018 November-December; 10(8):1226-1235

### SEQUENCES

The following sequences form part of the disclosure of the present application. A WIPO ST 25 compatible electronic sequence listing is provided with this application, too. For the avoidance of doubt, if discrepancies exist between the sequences in the following table and the electronic sequence listing, the sequences in this table shall be deemed to be the correct ones.

| No | Sequence | length | Position | Type | Origin |
|---|---|---|---|---|---|
| 1 | FNISVYMMH | 9 | 28-36 | HCDR1 of | 79D |
| | | | | anti CD117 | |
| 2 | WVASIYPYSGYTYY | 14 | 48-61 | HCDR2 of | 79D |
| | | | | anti CD117 | |
| 3 | RYVYHALDY | 9 | 99-107 | HCDR3 of | 79D |
| | | | | anti CD117 | |
| 4 | QRGLRNVAVA | 10 | 27-36 | LCDR1 of | 79D |
| | | | | anti CD117 | |
| 5 | LLIYSASSLYS | 11 | 48-58 | LCDR2 of | 79D |
| | | | | anti CD117 | |
| 6 | QQWAVHSLI | 9 | 91-99 | LCDR3 of | 79D |
| | | | | anti CD117 | |
| 7 | YTFTRYTMH | 9 | 27-35 | HCDR1 of | BlinCD3 |
| | | | | anti CD3 | |
| 8 | WIGYINPSRGYTNY | 14 | 47-60 | HCDR2 of | BlinCD3 |
| | | | | anti CD3 | |
| 9 | RYYDDHYCLDY | 11 | 98-108 | HCDR3 of | BlinCD3 |
| | | | | anti CD3 | |
| 10 | SSVSYMN | 7 | 27-33 | LCDR1 of | BlinCD3 |
| | | | | anti CD3 | |
| 11 | RWIYDTSKVAS | 11 | 41-55 | LCDR2 of | BlinCD3 |
| | | | | anti CD3 | |
| 12 | QQWSSNPL | 8 | 88-95 | LCDR3 of | BlinCD3 |
| | | | | anti CD3 | |
| 13 | YTFTRYTMH | 9 | 27-35 | HCDR1 of | Okt3 |
| | | | | anti CD3 | |
| 14 | WIGYINPSRGYTNY | 14 | 47-60 | HCDR2 of | Okt3 |
| | | | | anti CD3 | |
| 15 | RYYDDHYCLDY | 11 | 98-108 | HCDR3 of | Okt3 |
| | | | | anti CD3 | |
| 16 | SSVSYMN | 7 | 27-33 | LCDR1 of | Okt3 |
| | | | | anti CD3 | |
| 17 | RWIYDTSKLAS | 11 | 45-55 | LCDR2 of | Okt3 |
| | | | | anti CD3 | |
| 18 | QQWSSNPF | 8 | 88-95 | LCDR3 of | Okt3 |
| | | | | anti CD3 | |
| 19 | SEVQLVESGGGLVQPGGSLRLSCAASGFNISVYMMHWVRQAPGKGLEWVASIYPY | 118 | | HCVD of | 79D |
| | SGYTYYADSVKGRFTISADTSKNTAYLQMNSLRAEDTAVYYCARYVYHALDYWGQ | | | anti CD117 | |
| | GTLVTVSS | | | | |
| 20 | DIQMTQSPSSLSASVGDRVTITCRASQRGLRNVAVAWYQQKPGKAPKLLIYSASS | 111 | | LCVD of | 79D |
| | LYSGVPSRFSGSRSGTDFTLTISSLQPEDFATYYCQQWAVHSLITFGQGTKVEIKR | | | anti CD117 | |
| 21 | DIKLQQSGAELARPGASVKMSCKTSGYTFTRYTMHWVKQRPGQGLEWIGYINPSR | 119 | | HCVD of | BlinCD3 |
| | GYTNYNQKFKDKATLTTDKSSSTAYMQLSSLTSEDSAVYYCARYYDDHYCLDYWGQ | | | anti CD3 | |
| | GTTLTVSS | | | | |
| 22 | DIQLTQSPAIMSASPGEKVTMTCRASSSVSYMNWYQQKSGTSPKRWIYDTSKVAS | 106 | | LCVD of | BlinCD3 |
| | GVPYRFSGSGSGTSYSLTISSMEAEDAATYYCQQWSSNPLTFGAGTKLELK | | | anti CD3 | |
| 23 | QVQLQQSGAELARPGASVKMSCKASGYTFTRYTMHWVKQRPGQGLEWIGYINPS | 119 | | HCVD of | Okt3 |
| | RGYTNYNQKFKDKATLTTDKSSSTAYMQLSSLTSEDSAVYYCARYYDDHYCLDYWG | | | anti CD3 | |
| | QGTTLTVSS | | | | |
| 24 | QIVLTQSPAIMSASPGEKVTMTCSASSSVSYMNWYQQKSGTSPKRWIYDTSKLASG | 107 | | LCVD of | Okt3 |
| | VPAHFRGSGSGTSYSLTISGMEAEDAATYYCQQWSSNPFTFGSGTKLEINR | | | anti CD3 | |
| 25 | DIQMTQSPSSLSASVGDRVTITCRASQRGLRNVAVAWYQQKPGKAPKLLIYSASS | 491 | | Full length | 79Dx |
| | LYSGVPSRFSGSRSGTDFTLTISSLQPEDFATYYCQQWAVHSLITFGQGTKVEIKRG | | | BiTE | BlinCD3 |
| | GGGSGGGGSGGGGSEVQLVESGGGLVQPGGSLRLSCAASGFNISVYMMHWVRQAP | | | (including | |
| | GKGLEWVASIYPYSGYTYYADSVKGRFTISADTSKNTAYLQMNSLRAEDTAVYYCAR | | | linkers) | |
| | YVYHALDYWGQGTLVTVSSGGGGSDIKLQQSGAELARPGASVKMSCKTSGYTFTR | | | 79DxBlinCD3 | |
| | YTMHWVKQRPGQGLEWIGYINPSRGYTNYNQKFKDKATLTTDKSSSTAYMQLSSL | | | | |
| | TSEDSAVYYCARYYDDHYCLDYWGQGTTLTVSSVEGGSGGSGGSGGSGGVDDIQL | | | | |
| | TQSPAIMSASPGEKVTMTCRASSSVSYMNWYQQKSGTSPKRWIYDTSKVASGVPY | | | | |
| | RFSGSGSGTSYSLTISSMEAEDAATYYCQQWSSNPLTFGAGTKLELK | | | | |
| 26 | DIQMTQSPSSLSASVGDRVTITCRASQRGLRNVAVAWYQQKPGKAPKLLIYSASS | 492 | | Full length | 79Dx |
| | LYSGVPSRFSGSRSGTDFTLTISSLQPEDFATYYCQQWAVHSLITFGQGTKVEIKRG | | | BiTE | BlinCD3 |
| | GGGSGGGGSGGGGSEVQLVESGGGLVQPGGSLRLSCAASGFNISVYMMHWVRQAP | | | (including | |
| | GKGLEWVASIYPYSGYTYYADSVKGRFTISADTSKNTAYLQMNSLRAEDTAVYYCAR | | | linkers) | |
| | YVYHALDYWGQGTLVTVSSGGGGSQVQLQQSGAELARPGASVKMSCKASGYTFT | | | 79DxOKT3 | |
| | RYTMHWVKQRPGQGLEWIGYINPSRGYTNYNQKFKDKATLTTDKSSSTAYMQLSS | | | | |
| | LTSEDSAVYYCARYYDDHYCLDYWGQGTTLTVSSVEGGSGGSGGSGGSGGVDQIV | | | | |
| | LTQSPAIMSASPGEKVTMTCSASSSVSYMNWYQQKSGTSPKRWIYDTSKLASGVP | | | | |
| | AHFRGSGSGTSYSLTISGMEAEDAATYYCQQWSSNPFTFGSGTKLEINR | | | | |
| 27 | QVQLQQSGAELARPGASVKMSCKASGYTFTRYTMHWVKQRPGQGLEWIGYINPS | 244 | | scFV | Okt3 |
| | RGYTNYNQKFKDKATLTTDKSSSTAYMQLSSLTSEDSAVYYCARYYDDHYCLDYWG | | | sequence of | |
| | QGTTLTVSSVEGGSGGSGGSGGSGGVDQIVLTQSPAIMSASPGEKVTMTCSASSSV | | | anti CD3 | |
| | SYMNWYQQKSGTSPKRWIYDTSKLASGVPAHFRGSGSGTSYSLTISGMEAEDAAT | | | (VH-Linker- | |
| | YYCQQWSSNPFTFGSGTKLEINR | | | VL) | |
| 28 | DIKLQQSGAELARPGASVKMSCKTSGYTFTRYTMHWVKQRPGQGLEWIGYINPSR | 243 | | scFV | BlinCD3 |
| | GYTNYNQKFKDKATLTTDKSSSTAYMQLSSLTSEDSAVYYCARYYDDHYCLDYWGQ | | | sequence of | |
| | GTTLTVSSVEGGSGGSGGSGGSGGVDDIQLTQSPAIMSASPGEKVTMTCRASSSVS | | | anti CD3 | |
| | YMNWYQQKSGTSPKRWIYDTSKVASGVPYRFSGSGSGTSYSLTISSMEAEDAATYY | | | (VH-Linker- | |
| | CCKIWSSNPLTFGAGTKLELK | | | VL) | |
| 29 | DIQMTQSPSSLSASVGDRVTITCRASQRGLRNVAVAWYQQKPGKAPKLLIYSASS | 243 | | scFV | 79D |
| | SLYGVPSRFSGSRSGTDFTLTISSLQPEDFATYYCQQWAVHSLITFGQGTKVEIKRG | | | sequence of | |
| | GGGSGGGGSGGGGSEVQLVESGGGLVQPGGSLRLSCAASGFNISVYMMHWVRQAP | | | anti CD117 | |
| | GKGLEWVASIYPYSGYTYYADSVKGRFTISADTSKNTAYLQMNSLRAEDTAVYYCAR | | | (VL-Linker- | |
| | YVYHALDYWGQGTLVTVSS | | | VH) | |
| 30 | MGWSLILLFLVAVATGVHS | 19 | | Signal | |
| | | | | peptide | |
| 31 | GGGGSGGGGSGGGG | 14 | | N-terminal | |
| | | | | linker | |
| | | | | ((G₄S)₂G₄) | |
| 32 | GGGGS | 5 | | inner linker | |
| | | | | (G₄S) | |
| 33 | VEGGSGGSGGSGGSGGVD | 18 | | C-terminal | |
| | | | | linker | |
| | | | | (VE(GGS)₄G | |
| | | | | GVD) | |
| 34 | MGWSLILLFLVAVATGVHSDIQMTQSPSSLSASVGDRVTITCRASQRGLRNVAVA | 516 | | Full length | 79Dx |
| | WYQQKPGKAPKLLIYSASSLYSGVPSRFSGSRSGTDFTLTISSLQPEDFATYYCQQWA | | | BiTE | BlinCD3 |
| | VHSLITFGQGTKVEIKRGGGGSGGGGSGGGGSEVQLVESGGGLVQPGGSLRLSCA | | | (including | |
| | ASGFNISVYMMHWVRQAPGKGLEWVASIYPYSGYTYYADSVKGRFTISADTSKNT | | | signal | |
| | AYLQMNSLRAEDTAVYYCARYVYHALDYWGQGTLVTVSSGGGGSDIKLQQSGAEL | | | peptide, | |
| | ARPGASVKMSCKTSGYTFTRYTMHWVKQRPGQGLEWIGYINPSRGYTNYNQKFK | | | linkers and | |
| | DKATLTTDKSSSTAYMQLSSLTSEDSAVYYCARYYDDHYCLDYWGQGTTLTVSSVE | | | His tag) | |
| | GGSGGSGGSGGSGGVDDIQLTQSPAIMSASPGEKVTMTCRASSSVSYMNWYQQK | | | 79DxBlinCD3 | |
| | SGTSPKRWIYDTSKVASGVPYRFSGSGSGTSYSLTISSMEAEDAATYYCQQWSSNPL | | | | |
| | TFGAGTKLELKHHHHHH | | | | |
| 35 | GFNISVY | 7 | 28-36 | HCDR1 of | 79D |
| | | | | anti CD117 | |
| 36 | YPYSGY | 6 | 48-61 | HCDR2 of | 79D |
| | | | | anti CD117 | |
| 37 | YVYHALDY | 8 | 99-107 | HCDR3 of | 79D |
| | | | | anti CD117 | |
| 38 | RASQRGLRNVAVA | 13 | 27-36 | LCDR1 of | 79D |
| | | | | anti CD117 | |
| 39 | SASSLYS | 7 | 48-58 | LCDR2 of | 79D |
| | | | | anti CD117 | |
| 40 | QQWAVHSLIT | 10 | 91-99 | LCDR3 of | 79D |
| | | | | anti CD117 | |
| 41 | GYTFTRY | 7 | 27-35 | HCDR1 of | BlinCD3 |
| | | | | anti CD3 | |
| 42 | NPSRGY | 6 | 47-60 | HCDR2 of | BlinCD3 |
| | | | | anti CD3 | |
| 43 | YYDDHYCLDY | 10 | 98-108 | HCDR3 of | BlinCD3 |
| | | | | anti CD3 | |
| 44 | RASSSVSYMN | 10 | 27-33 | LCDR1 of | BlinCD3 |
| | | | | anti CD3 | |
| 45 | DTSKVAS | 7 | 41-55 | LCDR2 of | BlinCD3 |
| | | | | anti CD3 | |
| 46 | QQWSSNPLT | 9 | 88-95 | LCDR3 of | BlinCD3 |
| | | | | anti CD3 | |
| 47 | GFNISVY | 7 | 28-36 | HCDR1 of | 79D |
| | | | | anti CD117 | |
| 48 | YPYSGY | 6 | 48-61 | HCDR2 of | 79D |
| | | | | anti CD117 | |
| 49 | YVYHALDY | 8 | 99-107 | HCDR3 of | 79D |
| | | | | anti CD117 | |
| 50 | RASQRGLRNVAVA | 13 | 27-36 | LCDR1 of | 79D |
| | | | | anti CD117 | |
| 51 | SASSLYS | 7 | 48-58 | LCDR2 of | 79D |
| | | | | anti CD117 | |
| 52 | QQWAVHSLIT | 10 | 91-99 | LCDR3 of | 79D |
| | | | | anti CD117 | |
| 53 | GYTFTRY | 7 | 27-35 | HCDR1 of | BlinCD3 |
| | | | | anti CD3 | |
| 54 | NPSRGY | 6 | 47-60 | HCDR2 of | BlinCD3 |
| | | | | anti CD3 | |
| 55 | YYDDHYCLDY | 10 | 98-108 | HCDR3 of | BlinCD3 |
| | | | | anti CD3 | |
| 56 | RASSSVSYMN | 10 | 27-33 | LCDR1 of | BlinCD3 |
| | | | | anti CD3 | |
| 57 | DTSKVAS | 7 | 41-55 | LCDR2 of | BlinCD3 |
| | | | | anti CD3 | |
| 58 | QQWSSNPLT | 9 | 88-95 | LCDR3 of | BlinCD3 |
| | | | | anti CD3 | |
| 59 | DIQMTQSPSSLSASVGDRVTITCRASQRGLRNVAVAWYQQKPGKAPKLLIYSASSL | 497 | | Full length | 79Dx |
| | YSGVPSRFSGSRSGTDFTLTISSLQPEDFATYYCQQWAVHSLITFGQGTKVEIKRGG | | | BiTE | BlinCD3 |
| | GGSGGGGSGGGGSEVQLVESGGGLVQPGGSLRLSCAASGFNISVYMMHWVRQAP | | | (including | |
| | GKGLEWVASIYPYSGYTYYADSVKGRFTISADTSKNTAYLQMNSLRAEDTAVYYCAR | | | His Tag) | |
| | YVYHALDYWGQGTLVTVSSGGGGSDIKLQQSGAELARPGASVKMSCKTSGYTFTR | | | 79DxBlinCD3 | |
| | YTMHWVKQRPGQGLEWIGYINPSRGYTNYNQKFKDKATLTTDKSSSTAYMQLSSL | | | | |
| | TSEDSAVYYCARYYDDHYCLDYWGQGTTLTVSSVEGGSGGSGGSGGSGGVDDIQL | | | | |
| | TQSPAIMSASPGEKVTMTCRASSSVSYMNWYQQKSGTSPKRWIYDTSKVASGVPY | | | | |
| | RFSGSGSGTSYSLTISSMEAEDAATYYCQQWSSNPLTFGAGTKLELKHHHHHH | | | | |
| 60 | DIQMTQSPSSLSASVGDRVTITCRASQRGLRNVAVAWYQQKPGKAPKLLIYSASSL | 498 | | Full length | 79Dx |
| | YSGVPSRFSGSRSGTDFTLTISSLQPEDFATYYCQQWAVHSLITFGQGTKVEIKRGG | | | BiTE | Okt3 |
| | GGSGGGGSGGGGSEVQLVESGGGLVQPGGSLRLSCAASGFNISVYMMHWVRQAP | | | (including | |
| | GKGLEWVASIYPYSGYTYYADSVKGRFTISADTSKNTAYLQMNSLRAEDTAVYYCAR | | | His Tag) | |
| | YVYHALDYWGQGTLVTVSSGGGGSQVQLQQSGAELARPGASVKMSCKASGYTFT | | | 79DxOKT3 | |
| | RYTMHWVKQRPGQGLEWIGYINPSRGYTNYNQKFKDKATLTTDKSSSTAYMQLSS | | | | |
| | LTSEDSAVYYCARYYDDHYCLDYWGQGTTLTVSSVEGGSGGSGGSGGSGGVDQIV | | | | |
| | LTQSPAIMSASPGEKVTMTCSASSSVSYMNWYQQKSGTSPKRWIYDTSKLASGVP | | | | |
| | AHFRGSGSGTSYSLTISGMEAEDAATYYCQQWSSNPFTFGSGTKLEINRHHHHHH | | | | |
| 61 | SEVQLVESGGGLVQPGGSLRLSCAASGFNISVYMMHWVRQAPGKGLEWVASIYPY | 730 | | scDb-scFv | 79Dx |
| | SGYTYYADSVKGRFTISADTSKNTAYLQMNSLRAEDTAVYYCARYVYHALDYWGQ | | | | BlinCD3 |
| | GTLVTVSSGSSGGDIQMTQSPSSLSASVGDRVTITCRASQRGLRNVAVAWYQQKP | | | | |
| | GKAPKLLIYSASSLYSGVPSRFSGSRSGTDFTLTISSLQPEDFATYYCQQWAVHSLIT | | | | |
| | FGQGTKVEIKRGGGGSGGGGSGGGGSEVQLVESGGGLVQPGGSLRLSCAASGFNIS | | | | |
| | VYMMHWVRQAPGKGLEWVASIYPYSGYTYYADSVKGRFTISADTSKNTAYLQMN | | | | |
| | SLRAEDTAVYYCARYVYHALDYWGQGTLVTVSSGGSGGDIQMTQSPSSLSASVGD | | | | |
| | RVTITCRASQRGLRNVAVAWYQQKPGKAPKLLIYSASSLYSGVPSRFSGSRSGTDFTL | | | | |
| | TISSLQPEDFATYYCQQWAVHSLITFGQGTKVEIKRGGGGSDIKLQQSGAELARPGA | | | | |
| | SVKMSCKTSGYTFTRYTMHWVKQRPGQGLEWIGYINPSRGYTNYNQKFKDKATLT | | | | |
| | TDKSSSTAYMQLSSLTSEDSAVYYCARYYDDHYCLDYWGQGTTLTVSSVEGGSGGS | | | | |
| | GGSGGSGGVDDIQLTQSPAIMSASPGEKVTMTCRASSSVSYMNWYQQKSGTSPK | | | | |
| | RWIYDTSKVASGVPYRFSGSGSGTSYSLTISSMEAEDAATYYCQQWSSNPLTFGAG | | | | |
| | TKLELK | | | | |
| 62 | DIQLTQSPAIMSASPGEKVTMTCRASSSVSYMNWYQQKSGTSPKRWIYDTSKVAS | 472 | | TandAb | 79Dx |
| | GVPYRFSGSGSGTSYSLTISSMEAEDAATYYCQQWSSNPLTFGAGTKLELKGGSGGS | | | (2:2) | BlinCD3 |
| | SEVQLVESGGGLVQPGGSLRLSCAASGFNISVYMMHWVRQAPGKGLEWVASIYPY | | | | |
| | SGYTYYADSVKGRFTISADTSKNTAYLQMNSLRAEDTAVYYCARYVYHALDYWGQ | | | | |
| | GILVTVSSGGSGGSDIQMTQSPSSLSASVGDRVTITCRASQRGLRNVAVAWYQQK | | | | |
| | PGKAPKWYSASSLYSGVPSRFSGSRSGTDFTLTISSLOPEDFATYYCQQWAVHSLIT | | | | |
| | FGQGTKVEIKRGGSGGSDIKLQQSGAELARPGASVKMSCKTSGYTFTRYTMHWVK | | | | |
| | QRPGQGLEWIGYINPSRGYTNYNQKFKDKATLTTDKSSSTAYMQLSSLTSEDSAVYY | | | | |
| | CARYYDDHYCLDYWGQGTTLTVSS | | | | |
| 63 | DIQLTQSPAIMSASPGEKVTMTCRASSSVSYMNWYQQKSGTSPKRWIYDTSKVAS | 231 | | Dart Chain | 79Dx |
| | GVPYRFSGSGSGTSYSLTISSMEAEDAATYYCQQWSSNPLTFGAGTKLELKGGSGGS | | | 1 | BlinCD3 |
| | SEVQLVESGGGLVQPGGSLRLSCAASGFNISVYMMHWVRQAPGKGLEWVASIYPY | | | | |
| | SGYTYYADSVKGRFTISADTSKNTAYLQMNSLRAEDTAVYYCARYVYHALDYWGQ | | | | |
| | GTLVTVSSC | | | | |
| 64 | DIQMTQSPSSLSASVGDRVTITCRASQRGLRNVAVAWYQQKPGKAPKLLIYSASSLY | 237 | | Dart Chain | 79Dx |
| | SGVPSRFSGSRSGTDFTLTISSLQPEDFATYYCQQWAVHSLITFGQGTKVEIKRGGSG | | | 2 | BlinCD3 |
| | GSDIKLQQSGAELARPGASVKMSCKTSGYTFTRYTMHWVKQRPGQGLEWIGYINP | | | | |
| | SRGYTNYNQKFKDKATLTTDKSSSTAYMQLSSLTSEDSAVYYCARYYDDHYCLDYW | | | | |
| | GQGTTLTVSSC | | | | |

What is claimed is:

1. A bispecific binding agent comprising at least a first binding domain and a second binding domain, wherein said first binding domain binds to CD117/c-KIT and wherein said second binding domain binds to CD3.

2. The bispecific binding agent according to claim 1, wherein said first binding domain binds to the extracellular domain of CD117/c-KIT and/or said second binding domain binds to the ε chain of CD3 and/or the γ chain of CD3.

3. The bispecific binding agent according to any one of the aforementioned claims, which is in the format of a full-length antibody or an antibody fragment.

4. The bispecific binding agent according to any one of the aforementioned claims, which is in the format of at least one selected from the group consisting of
DVD-Ig
Knobs in Holes
Crossmab
BiTE™
Nanobody
Diabody
DART™
TandAb™′ and/or
scDb-scFv

5. The bispecific binding agent according to any one of the aforementioned claims, wherein the first binding domain which binds to CD117/c-KIT
a) comprises a set of heavy chain/light chain complementarity determining regions (CDR) comprised in the heavy chain/light variable region sequence pair of the anti CD117 antibody 79D, as set forth in SEQ ID NOs 19 and 20
b) comprises a set of heavy chain/light chain complementarity determining regions (CDR) comprising the following sequences
HC CDR1 (SEQ ID NO 1 or 35)
HC CDR2 (SEQ ID NO 2 or 36)
HC CDR3 (SEQ ID NO 3 or 37)
LC CDR1 (SEQ ID NO 4 or 38)
LC CDR2 (SEQ ID NO 5 or 39), and
LC CDR3 (SEQ ID NO 6 or 40)
c) comprises the heavy chain/light chain complementarity determining regions (CDR) of b), with the proviso that at least one of the CDRs has up to 3 amino acid substitutions relative to the respective SEQ ID NO 1-6, or SEQ ID NO 35-40, and/or
d) comprises the heavy chain/light chain complementarity determining regions (CDR) of b), with the proviso that at least one of the CDRs has a sequence identity of ≥66% to the respective SEQ ID NO 1-6, or SEQ ID NO 35-40,
wherein the CDRs are embedded in a suitable protein framework so as to be capable to bind to CD117/c-KIT.

6. The bispecific binding agent according to any one of the aforementioned claims, wherein the second binding domain which binds to CD3
a) comprises a set of heavy chain/light chain complementarity determining regions (CDR) comprising the following sequences OKT3, BlinCD3
b) comprises a set of heavy chain/light chain complementarity determining regions (CDR) comprised in the heavy chain/light variable region sequence pair of one of the anti CD3 antibodies BlinCD3, OKT3, and [ . . . ], as set forth in SEQ ID NOs 21 and 22, or SEQ ID NO 23 and 24
c) comprises the set of heavy chain/light chain complementarity determining regions (CDR) comprising the following sequences
HC CDR1 (SEQ ID NO 7 or 41)
HC CDR2 (SEQ ID NO 8 or 42)
HC CDR3 (SEQ ID NO 9 or 43)
LC CDR1 (SEQ ID NO 10 or 44)
LC CDR2 (SEQ ID NO 11 or 45), and
LC CDR3 (SEQ ID NO 12 or 46)
d) comprises the set of heavy chain/light chain complementarity determining regions (CDR) comprising the following sequences
HC CDR1 (SEQ ID NO 13)
HC CDR2 (SEQ ID NO 14)
HC CDR3 (SEQ ID NO 15)
LC CDR1 (SEQ ID NO 16)
LC CDR2 (SEQ ID NO 17), and
LC CDR3 (SEQ ID NO 18)
e) comprises the heavy chain/light chain complementarity determining regions (CDR) of b) or c), with the proviso that at least one of the CDRs has up to 3 amino acid substitutions relative to the respective SEQ ID NO 7-12 or SEQ ID NO 41 or SEQ ID NO 13-18, and/or
f) comprises the heavy chain/light chain complementarity determining regions (CDR) of b), with the proviso that at least one of the CDRs has a sequence identity of ≥66% to the respective SEQ ID NO 7-12 or SEQ ID NO 41 or SEQ ID NO 13-18,
wherein the CDRs are embedded in a suitable protein framework so as to be capable to bind to CD3.

7. The bispecific binding agent according to any one of the aforementioned claims, wherein the framework is a human VH/VL framework.

8. The bispecific binding agent according to any one of the aforementioned claims, wherein the first binding domain which binds to CD117/c-KIT comprises
a) the variable domains of of the antibody 79D
b) the heavy chain/light chain variable domains (VD)
HC VD (SEQ ID NO 19), and
LC VD (SEQ ID NO 20)
c) the heavy chain/light chain variable domains (VD) of b), with the proviso that
the HCVD has a sequence identity of ≥80% to the respective SEQ ID NO 19, and/or
the LCDVD has a sequence identity of ≥80% to the respective SEQ ID NO 20,
d) the heavy chain/light chain variable domains (VD) of b), with the proviso that at least one of the HCVD or LCVD has up to 10 amino acid substitutions relative to the respective SEQ ID NO 19 and/or 20,
said bispecific binding agent still being capable to bind to CD117/c-KIT.

9. The bispecific binding agent according to any one of the aforementioned claims, wherein the second binding domain which binds to CD3 comprises
a) the variable domains of one antibody selected from the group consisting of OKT3 and BlinCD3
b) the heavy chain/light chain variable domains (VD)
HC VD (SEQ ID NO 21 or 23), and
LC VD (SEQ ID NO 22 or 24)
c) the heavy chain/light chain variable domains (VD) of b), with the proviso that
the HCVD has a sequence identity of ≥80% to the respective SEQ ID NO 21 or 23, and/or
the LCDVD has a sequence identity of ≥80% to the respective SEQ ID NO 22 or 24
d) the heavy chain/light chain variable domains (VD) of b), with the proviso that at least one of the HCVD or LCVD has up to 10 amino acid substitutions relative to the respective SEQ ID NOs,
said bispecific binding agent still being capable to bind to CD3.

10. The bispecific binding agent according to any one of the aforementioned claims to any one of the aforementioned claims, wherein at least one amino acid substitution is a conservative amino acid substitution.

11. The bispecific binding agent according to any one of the aforementioned claims, wherein the corresponding variable heavy chain regions (VH) and the corresponding variable light chain regions (VL) regions are arranged, from N-terminus to C-terminus, in the order,
V_{L}(CD117)-Linker-V_{H}(CD117)-Linker-V_{H}(CD3)-Linker-V_{L}(CD3),
V_{L}(CD117)-Linker-V_{H}(CD117)-Linker-V_{L}(CD3)-Linker-V_{H}(CD3),
V_{H}(CD117)-Linker-V_{L}(CD117)-Linker-V_{H}(CD3)-Linker-V_{L}(CD3),
V_{H}(CD117)-Linker-V_{L}(CD117)-Linker-V_{L}(CD3)-Linker-V_{H}(CD3),
V_{L}(CD3)-Linker-V_{H}(CD3)-Linker-V_{H}(CD117)-Linker-V_{L}(CD117),
V_{L}(CD3)-Linker-V_{H}(CD3)-Linker-V_{L}(CD117)-Linker-V_{H}(CD117),
V_{H}(CD3)-Linker-V_{L}(CD3)-Linker-V_{H}(CD117)-Linker-V_{L}(CD117),
V_{H}(CD3)-Linker-V_{L}(CD3)-Linker-V_{L}(CD117)-Linker-V_{H}(CD117),
wherein the term “Linker” defines an optional polypeptide linker.

12. The bispecific binding agent according to any one of the aforementioned claims, wherein the second binding domain which binds to CD3
a) comprises a scFv-like sequence as set forth in any one of SEQ ID NOs 28 or 29, or
b) comprises an amino acid sequence that has a sequence identity of ≥80% to any one of SEQ ID NOs 28 or 29.

13. The bispecific binding agent according to any one of the aforementioned claims, wherein the first binding domain which binds to CD117/cKIT
a) comprises a scFv-like sequence as set forth in SEQ ID NO 27, or
b) comprises an amino acid sequence that has a sequence identity of ≥80% to SEQ ID NOs 27.

14. The bispecific binding agent according to any one of the aforementioned claims, which
a) comprises at least one of the sequences selected from SEQ ID NO 25, 26, 34, 59 or 60, or
b) comprises an amino acid sequence that has a sequence identity of ≥80% to SEQ ID NO 25, 26, 34, 59 or 60.

15. The bispecific binding agent according to any one of the aforementioned claims, which agent has at least one of
target binding affinity of ≥50% to CD3, and measured by SPR, and/or
target binding affinity of ≥50% to CD117/c-KIT, and measured by SPR,
compared to that of the bispecific binding agent according to claim 14.

16. A bispecific binding agent that competes for binding to CD117/c-KIT and CD3 with the bispecific binding agent according to claim 14.

17. A bispecific binding agent that binds to essentially the same, or the same, epitopes on CD3 and CD117/c-KIT as the bispecific binding agent according to claim 14.

18. A nucleic that encodes for a binding agent according to any one of the aforementioned claims.

19. A pharmaceutical composition comprising a bispecific binding agent or a nucleic acid according to any one of the aforementioned claims, and optionally one or more pharmaceutically acceptable excipients.

20. A combination comprising (i) the bispecific binding agent or pharmaceutical composition according to any one of the aforementioned claims and (ii) one or more therapeutically active compounds.

21. The bispecific binding agent, pharmaceutical composition or combination according to any one of the aforementioned claims for use (in the manufacture of a medicament) in the treatment of a human or animal subject
being diagnosed for,
suffering from or
being at risk of
developing a neoplastic disease, or for the prevention of such condition.

22. A method for treating or preventing a neoplastic disease, comprising administering to a subject in need thereof an effective amount of the bispecific binding agent, pharmaceutical composition or combination according to any one of the aforementioned claims

23. A therapeutic kit of parts comprising:
a) the bispecific binding agent, pharmaceutical composition or combination according to any one of the aforementioned claims
b) an apparatus for administering the composition, composition or combination, and
c) instructions for use.
